(19) United States
(12) Patent Application Publication
(10) Pub. No.: US 2025/0131748 A1
(43) Pub. Date: Apr. 24, 2025

(54) SYSTEMS AND METHODS FOR POLYMERASE CHAIN REACTION QUANTIFICATION

(71) Applicant: Combinati Incorporated, Carlsbad, CA (US)

(72) Inventors: Roger DETTLOFF, Palo Alto, CA (US); Steve GALLAGHER, Underwood, WA (US)

(21) Appl. No.: 18/691,571

(22) PCT Filed: Sep. 14, 2022
(86) PCT No.: PCT/US2022/043548

§ 371 Date: Mar. 13, 2024

Related U.S. Application Data
(60) Provisional application No. 63/244,237, filed on Sep. 14, 2021.
(60) Provisional application No. 63/244,238, filed on Sep. 14, 2021.
(60) Provisional application No. 63/244,684, filed on Sep. 15, 2021.

(51) Int. Cl. G06V 20/69; G06T 5/50; G06T 7/00; G06T 7/13; G06T 7/32; G06T 7/33; G06T 7/73; G06V 10/764; G06V 10/774; G06V 10/82; G16B 25/20

(52) U.S. Cl. CPC G06V 20/698; G06T 5/50; G06T 7/0016; G06T 7/13; G06T 7/32; G06T 7/337; G06T 7/74; G06V 10/764; G06V 10/774; G06V 10/82; G16B 25/20; G06T 2207/10064; G06T 2207/20081; G06T 2207/20084; G06T 2207/20164; G06T 2207/20224; G06T 2207/30072; G06V 2201/03; G06V 2201/04

(57) ABSTRACT

Methods and systems are disclosed for quantifying one or more target concentrations in a biological sample using an analyte detection apparatus configured to analyze an array of partitions of the biological sample. A disclosed method comprises calculating expected locations of partitions in a representation of the array of partitions such as an image, based on corner locations of the array of partitions and analyzing images representing partitions associated with the expected locations of the partitions. The method further comprises determining observed locations of the partitions based on an analysis result of the images and quantifying the one or more target concentrations in the biological sample based on the observed locations of the partitions. These and other methods and systems are disclosed herein.

### CROSS-REFERENCE TO RELATED APPLICATIONS

This application claims the benefit of the following U.S. Provisional Applications 63/244,237, filed on Sep. 14, 2021; 63/244,238, filed on Sep. 14, 2021; and 63/244,684, filed on Sep. 15, 2021. To the extent permitted in applicable jurisdictions, the entire contents of these applications are incorporated herein by reference.

### TECHNICAL FIELD

This disclosure relates generally to monitoring, measuring, and/or analyzing biological and biochemical reactions, and more specifically to inventive methods of polymerase chain reaction analysis for quantifying target concentrations in a biological sample.

### BACKGROUND

Quantitative detection and analysis methods such as the polymerase chain reaction (PCR) is a technique used to quantify biological molecules in a biological sample. Generally, PCR amplifies nucleic acids with the DNA polymerase enzyme responsible for forming new copies of DNA. Based on the theory that such amplification is exponential, a specific segment of DNA, e.g., nucleic acid molecule or nucleotide sequence, can be amplified millions or billions of times using PCR, producing enough copies to be analyzed using other techniques.

“Analog” quantification (e.g., in analog PCR) relies on extrapolating measurements based on measured patterns. For example, a target analyte may be quantified by comparing the number of amplification cycles and amount of PCR end-product to those of a reference sample. However, this type of quantification can be complicated by uncertainties and inaccuracies. Detection efficiency in a test sample may be different from that of reference samples. For example, in PCR, initial amplification cycles may not be exponential, and PCR amplification may plateau after an uncertain number of cycles. Particularly, low initial concentrations of target analytes may be missed completely when they do not amplify to detectable levels.

“Digital” quantification methods (e.g., digital PCR or dPCR) are a biotechnological refinement of analog methods offering more robust absolute quantification of analytes with higher accuracy and precision than analog methods. Digital quantification is more adept at detecting and quantifying concentrations of hard to detect rare targets, providing a more precise quantitation of samples or analysis (e.g., nucleotide sequences), and measuring low fold changes in analyte concentration. Consequently, digital quantification has many applications in basic research, clinical diagnostics, and environmental testing. For example, digital PCR has been applied to pathogen detection and cancer monitoring, copy number variation analysis, single gene expression analysis, rare sequence detection, gene expression profiling and single-cell analysis, detection of DNA contaminants in bioprocessing, validation of gene edits, and detection of specific methylation changes in DNA as biomarkers of cancer.

In contrast to an analog measurement that relies on extrapolating certain measurements based on measured patterns (e.g., exponential amplification cycles), digital quantification methods can quantitatively and discretely measure a certain analyte. Digital quantification can be performed on biological samples that contain or are suspected to contain a target analyte of interest, such as a cell, tissue, or specimen such as hair, a biological fluid such as blood, urine, saliva, etc., a cell cluster such as a microbial colony, or an organism, cell, microbe, bacterium, virus, protein, antibody, or nucleic acids such as such as DNA or RNA molecules. Target analytes include “original” analytes that were originally present in the biological sample as well any “synthetic” analytes that are indicative of the presence of original analytes which may be added or generated during detection, including PCR amplicons, antigen-antibody complexes, etc. Digital quantification (e.g., digital PCR) begins with a sample including a relatively small number of a target analyte, e.g., a polynucleotide or nucleotide sequence template DNA (or RNA). The sample is partitioned into a large number of smaller test samples, which will ideally contain either one target analyte or none of the target analytes such that a separate detection reaction can be carried out in each partition individually. Suitable partitions are individual targets that are sufficiently distanced from other individual targets to allow for individual detection or quantification, which may or may not be fluidically isolated from each other. Partitions may or may not include separating barriers such as walls or membranes or liquids that are immiscible with the sample, or semisolid media. Exemplary partitions include individually distanced targets, e.g., deposited on a substrate such as a glass slide, a tube, open or closed well, droplet, vesicle, chamber or bead, or any representation of an individual signal derived from a target that is distinguishable over background or noise, for example a bright spot over a darker background in a digital or analog image. In digital PCR methods, when the samples are thermally cycled using a PCR apparatus, the samples containing the target concentration are amplified and produce a positive detection signal, while the samples that do not contain the target concentration are not amplified and produce no detection signal. After multiple PCR amplification cycles, the samples are imaged and analyzed for fluorescence, which is used to quantify the target concentration in the samples.

### SUMMARY

Particular processes for quantifying target concentrations in a biological sample using digital quantification, including processes for identifying the precise locations of each of the partitions, determining which partitions to accept for analysis, and interpreting the signal values in a representation (e.g., image) of the analyzed partitions, can present a variety of technical challenges that can adversely affect the goal of obtaining useful test results.

Various computer-implemented systems, methods, and articles of manufacture for quantifying one or more target concentrations in a biological sample using an analyte detection (e.g., a PCR) apparatus, and for training a machine-learning model used for analyzing one or more biological samples by an analyte detection (e.g., a PCR) apparatus, are described herein.

In one embodiment, a method for quantifying one or more target concentrations in a biological sample using an analyte detection (e.g., a PCR) apparatus configured to analyze an array of partitions of the biological sample is provided. The method comprises obtaining an image representing the array of partitions disposed in a container. The method further comprises determining, based on the image representing the array of partitions, a location associated with a plurality of corners of the array of partitions; and quantifying, based on the location data associated with the plurality of corners, a first target concentration in the biological sample.

In one embodiment, a method of quantifying one or more target concentrations in a biological sample using an analyte detection (e.g., a PCR) apparatus configured to analyze an array of partitions (e.g., an array of about 1000-5000, 10,000-50,000, about 100,000, 1,000,000 or 100,000,000) of the biological sample is provided. The method comprises calculating expected locations of partitions in a representation of the array of partitions such as an image, based on corner locations of the array of partitions and analyzing images representing partitions associated with the expected locations of the partitions. The method further comprises determining observed locations of the partitions based on an analysis result of the images and quantifying the one or more target concentrations in the biological sample based on the observed locations of the partitions.

In one embodiment, a method of training a machine-learning model used for analyzing one or more biological samples by an analyte detection (e.g., a PCR) apparatus is provided. The method is performed by one or more computing devices and comprises obtaining a first plurality of images identified as positive partition images and obtaining a second plurality of images identified as non-positive partition images. The second plurality of images comprises one or more images modified from one or more other images identified as non-positive partition images. The method further comprises generating one or more datasets using the first plurality of images and the second plurality of images and determining, by the one or more computing devices, a set of parameters of the machine-learning model by training the machine-learning model using at least one of the one or more datasets. A trained machine-learning model is configured based on the set of parameters to analyze one or more target concentrations in the one or more biological samples.

In one embodiment, a method for quantifying one or more target concentrations in a biological sample using an analyte detection (e.g., a PCR) apparatus configured to analyze an array of partitions of the biological sample is provided. The method comprises providing a plurality of partition images to a trained machine-learning model. The plurality of partition images represents a corresponding plurality of partitions that are at least a subset of the array of partitions. The method further comprises classifying, by the trained machine-learning model, the plurality of partition images as positive partition images or non-positive partition images. The trained machine-learning model is trained by using one or more images modified from one or more other images identified as non-positive partition images. The method further comprises quantifying the one or more target concentrations in the biological sample based on a classification result.

Various objects, features, aspects, and advantages of the inventive subject matter will become more apparent from the following specification, along with the accompanying drawings in which like numerals represent like components.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates a block diagram of a PCR apparatus and an array of partitions disposed in a microfluidic array plate.

FIG. 2 illustrates a plurality of exemplary pre-PCR images and post-PCR images representing one or more arrays of partitions disposed in a microfluidic array plate.

FIG. 3 illustrates an exemplary functional block diagram of an analysis pipeline for quantifying one or more target concentrations in a biological sample using a PCR apparatus.

FIG. 4A illustrates an image of an array of partitions and template images used to determine corner locations of the array.

FIGS. 4B and 4C illustrate exemplary visual indications of a match between a template image and an edge.

FIG. 5A illustrates an array of partitions represented in an image.

FIG. 5B illustrates a zoomed-in image representing a corner area of the array in FIG. 5A.

FIGS. 6A-6C illustrate images showing deviations of observed locations of partitions from the expected locations.

FIG. 6D illustrates an error map visually displaying deviations of the observed locations from the expected locations and the directions of the deviations.

FIG. 6E illustrates a diagram of magnitudes of location errors with respect to the number of partitions in an array of partitions.

FIGS. 7A-7C illustrates pre- and post-PCR images and image subtraction using the pre- and post-PCR images.

FIG. 8A illustrates examples of positive partition images.

FIG. 8B illustrates examples of non-positive partition images.

FIG. 9A illustrates a diagram of partition signals obtained before a classification using a machine-learning model is performed.

FIG. 9B illustrates a diagram of partition signal obtained after a classification using a machine-learning model is performed.

FIG. 10A illustrates an image representing partitions with relatively small location errors.

FIG. 10B illustrates an image representing partitions with relatively large location errors.

FIG. 11A illustrates diagrams of fluorescence signals obtained before spectral compensation is applied.

FIG. 11B illustrates diagrams of fluorescence signals obtained after spectral compensation is applied.

FIG. 12A illustrates an exemplary method of processing the summaries of signals using a mixing model such as a Gaussian Mixture Model (GMM).

FIGS. 12B-12E are exemplary diagrams of fluorescence signals clustered by a GMM clustering method.

FIG. 13 is a flowchart illustrating a method for determining corner locations of an array of partitions according to various embodiments.

FIG. 14 is a flowchart illustrating a method for determining observed locations of partitions according to various embodiments.

FIG. 15 is a flowchart illustrating a method for training a machine-learning model according to various embodiments.

FIG. 16 is a flowchart illustrating a method for classifying partition images according to various embodiments.

FIG. 17 illustrates a block diagram of a computer system that can be used for implementing one or more aspects of the various embodiments.

While the invention is described with reference to the above drawings, the drawings are intended to be illustrative, and other embodiments are consistent with the spirit, and within the scope, of the invention.

### DETAILED DESCRIPTION

To provide a more thorough understanding of the present invention, the following description sets forth numerous specific details, such as specific configurations, parameters, examples, and the like. It should be recognized, however, that such description is not intended as a limitation on the scope of the present invention but is intended to provide a better description of the exemplary embodiments.

Throughout the specification and claims, the following terms take the meanings explicitly associated herein, unless the context clearly dictates otherwise:

The phrase “in one embodiment” as used herein does not necessarily refer to the same embodiment, though it may. Thus, as described below, various embodiments of the invention may be readily combined, without departing from the scope or spirit of the invention.

As used herein, the term “or” is an inclusive “or” operator and is equivalent to the term “and/or,” unless the context clearly dictates otherwise.

The term “based on” is not exclusive and allows for being based on additional factors not described unless the context clearly dictates otherwise.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Within the context of a networked environment where two or more components or devices are able to exchange data, the terms “coupled to” and “coupled with” are also used to mean “communicatively coupled with”, possibly via one or more intermediary devices.

In addition, throughout the specification, the meaning of “a”, “an”, and “the” includes plural references, and the meaning of “in” includes “in” and “on”.

Although some of the various embodiments presented herein constitute a single combination of inventive elements, it should be appreciated that the inventive subject matter is considered to include all possible combinations of the disclosed elements. As such, if one embodiment comprises elements A, B, and C, and another embodiment comprises elements B and D, then the inventive subject matter is also considered to include other remaining combinations of A, B, C, or D, even if not explicitly discussed herein. Further, the transitional term “comprising” means to have as parts or members, or to be those parts or members. As used herein, the transitional term “comprising” is inclusive or open-ended and does not exclude additional, unrecited elements or method steps.

Throughout the following disclosure, numerous references may be made regarding servers, services, interfaces, engines, modules, clients, peers, portals, platforms, or other systems formed from computing devices. It should be appreciated that the use of such terms is deemed to represent one or more computing devices having at least one processor (e.g., ASIC, FPGA, DSP, x86, ARM, ColdFire, GPU, multi-core processors, etc.) configured to execute software instructions stored on a computer readable tangible, non-transitory medium (e.g., hard drive, solid state drive, RAM, flash, ROM, etc.). For example, a server can include one or more computers operating as a web server, database server, or other type of computer server in a manner to fulfill described roles, responsibilities, or functions. One should further appreciate the disclosed computer-based algorithms, processes, methods, or other types of instruction sets can be embodied as a computer program product comprising a non-transitory, tangible computer readable medium storing the instructions that cause a processor to execute the disclosed steps. The various servers, systems, databases, or interfaces can exchange data using standardized protocols or algorithms, possibly based on HTTP, HTTPS, AES, public-private key exchanges, web service APIs, known financial transaction protocols, or other electronic information exchanging methods. Data exchanges can be conducted over a packet-switched network, a circuit-switched network, the Internet, LAN, WAN, VPN, or other type of network.

As used in the description herein and throughout the claims that follow, when a system, engine, server, device, module, or other computing element is described as being configured to perform or execute functions on data in a memory, the meaning of “configured to” or “programmed to” is defined as one or more processors or cores of the computing element being programmed by a set of software instructions stored in the memory of the computing element to execute the set of functions on target data or data objects stored in the memory.

It should be noted that any language directed to a computer should be read to include any suitable combination of computing devices or network platforms, including servers, interfaces, systems, databases, agents, peers, engines, controllers, modules, or other types of computing devices operating individually or collectively. One should appreciate the computing devices comprise a processor configured to execute software instructions stored on a tangible, non-transitory computer readable storage medium (e.g., hard drive, FPGA, PLA, solid state drive, RAM, flash, ROM, etc.). The software instructions configure or program the computing device to provide the roles, responsibilities, or other functionality as discussed below with respect to the disclosed apparatus. Further, the disclosed technologies can be embodied as a computer program product that includes a non-transitory computer readable medium storing the software instructions that causes a processor to execute the disclosed steps associated with implementations of computer-based algorithms, processes, methods, or other instructions. In some embodiments, the various servers, systems, databases, or interfaces exchange data using standardized protocols or algorithms, possibly based on HTTP, HTTPS, AES, public-private key exchanges, web service APIs, known financial transaction protocols, or other electronic information exchanging methods. Data exchanges among devices can be conducted over a packet-switched network, the Internet, LAN, WAN, VPN, or other type of packet switched network; a circuit switched network; cell switched network; or other type of network.

In various embodiments, the devices, instruments, systems, and methods described herein may be used to detect one or more types of biological components of interest. These biological components of interest may be any suitable biological target including, but are not limited to, DNA sequences (including cell-free DNA), RNA sequences, genes, oligonucleotides, molecules, proteins, biomarkers, cells (e.g., circulating tumor cells), or any other suitable target biomolecule.

In various embodiments, such biological components may be used in conjunction with various digital PCR methods and systems in applications such as multiplex digital PCR, viral detection and quantification standards, genotyping, sequencing validation, mutation detection, detection of genetically modified organisms, fetal diagnostics, rare allele detection, and copy number variation.

Embodiments of the present disclosure are generally directed to devices, instruments, systems, and methods for measuring or quantifying a biological reaction for a large number of small volume samples.

While generally applicable to digital quantification such as PCR, it should be recognized that any other suitable quantification method may be used in accordance with various embodiments described herein. Suitable PCR methods include, but are not limited to, digital PCR, allele-specific PCR, asymmetric PCR, ligation-mediated PCR, multiplex PCR, nested PCR, qPCR, genome walking, and bridge PCR, for example.

As used herein, thermal cycling may include using a thermal cycler, isothermal amplification, thermal convention, infrared mediated thermal cycling, or helicase dependent amplification, for example.

According to various embodiments, detection of a target may be, but is not limited to, fluorescence detection, detection of positive or negative ions, pH detection, voltage detection, or current detection, alone or in combination, for example.

Various embodiments described herein are particularly suited for digital PCR (dPCR). In digital PCR, a solution containing a relatively small number of a target analyte, e.g., a polynucleotide or nucleotide sequence, may be subdivided into a large number of small test samples, such that each sample generally contains either one molecule of the target analyte, e.g., a nucleotide sequence, or none of the target. When the samples are subsequently thermally cycled in a PCR protocol, procedure, or experiment, the sample containing the target are amplified and produce a positive detection signal, while the samples containing no target are not amplified and produce no detection signal. Using Poisson statistics, the number of targets in the original solution may be correlated to the number of samples producing a positive detection signal.

One should appreciate that the disclosed techniques provide many advantageous technical effects including automated methods for quantifying one or more target concentrations in a biological sample using an analyte detection (e.g., a PCR) apparatus. The techniques described herein employ logic to automate various processes, including processes currently performed using manual human effort. Further, the disclosed techniques have been designed to support data accuracy and allow for processing data algorithms and complex permutations on a scale and speed that cannot be achieved using manual human effort.

It should also be appreciated that the following specification is not intended as an extensive overview, and as such, concepts may be simplified in the interests of clarity and brevity.

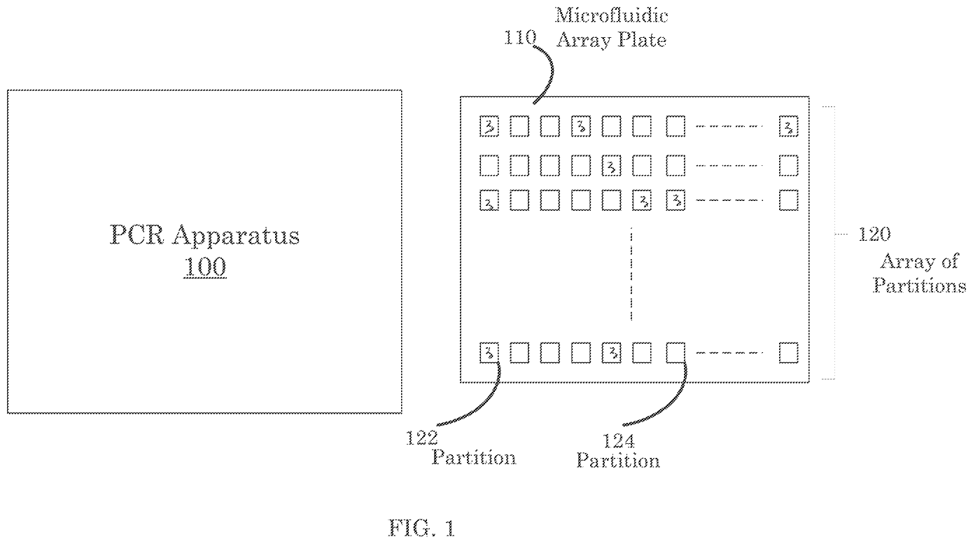

FIG. 1 illustrates a block diagram of a PCR apparatus 100 and a microfluidic array plate 110 having an array of partitions 120 disposed therein. In one embodiment, PCR apparatus 100 is a digital PCR. As described above, digital PCR (dPCR) uses a solution including a relatively small number of a target analyte, e.g., a polynucleotide or nucleotide sequence template DNA (or RNA), fluorescence-quencher probes, primers, and a PCR master mix comprising DNA polymerase and reaction buffers at optimal concentrations. The solution is partitioned into a large number of small test samples, e.g., tens of thousands of microchambers disposed within a microfluidic array plate 110. As shown in FIG. 1, microfluidic array plate 110 includes an array of microchambers formed by multiple rows and columns of microchambers (e.g., nano-liter sized microchambers). A microchamber may have nucleic acid binding surfaces for better binding with a target in the test sample. A small test sample is disposed in each microchamber of the array, thereby forming an array of partitions 120. A partition 122 thus includes a microchamber and a small test sample disposed therein. Typically, some partitions of array 120 include a test sample that has one or more targets (e.g., a target analyte such as a nucleotide sequence) and some partitions don't. In FIG. 1, for example, partition 122 includes a target but partition 124 doesn't include one. Thermal cycling is subsequently performed with respect to array of partitions 120 using PCR apparatus 100.

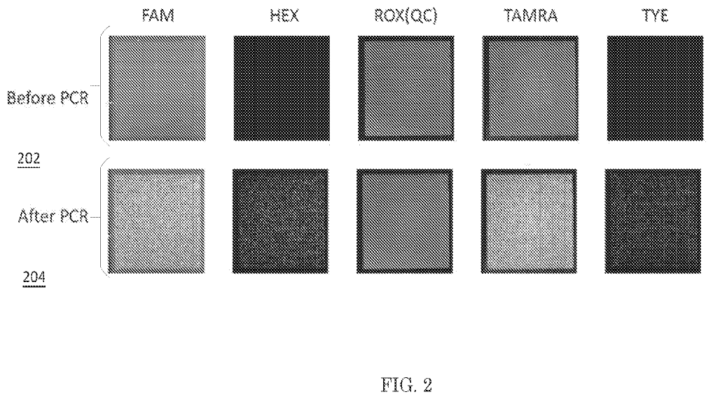

FIG. 2 illustrates a plurality of exemplary pre-PCR images 202 and post-PCR images 204 representing array of partitions 120 disposed in microfluidic array plate 110. In FIG. 2, images 202 are pre-PCR images representing array of partitions 120 before the PCR amplification of one or more targets in a biological sample is performed. Images 204 are post-PCR images representing array of partitions 120 after the amplification of one or more targets is performed. In one embodiment, images 202 and 204 are fluorescence images provided by multiple fluorescence channels of PCR apparatus 100.

PCR apparatus 100 can have multiplexing capabilities and is thus able to quantify multiple targets simultaneously. Multiplexing capabilities are obtained by having multiple fluorescence channels in PCR apparatus 100. In some embodiments, multiple fluorescence channels use different types of dyes for generating signals having different spectral wavelengths. Different dyes may each bind with a different target and produce signals with a different fluorescence color or spectrum. In FIG. 2, five exemplary types of dyes are used in different fluorescence channels in a PCR apparatus. The five different dyes may include a Carboxyfluorescein (FAM) based dye that produces signals having a blue fluorescence color, a Hexachloro-fluorescein (HEX) based dye that produces signals having a green fluorescence color, a 6-carboxy-X-rhodamine (ROX) based dye that produces signals having a red fluorescence color, a Tetramethylrhodamine (TAMRA) based dye that produces signals having a red or yellow fluorescence color, and a TYE™ based dye that produces a dark red fluorescence color. In some embodiments, the ROX based dye is used as a control reference dye.

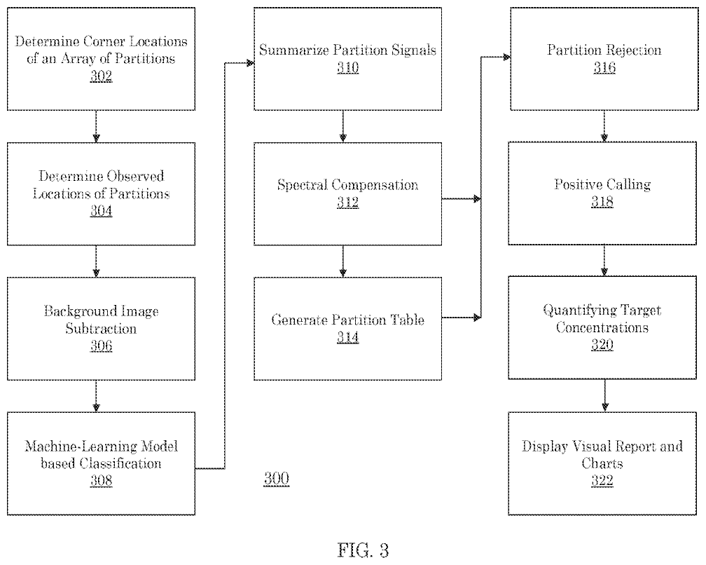

FIG. 3 illustrates an exemplary functional block diagram of an analysis pipeline 300 for quantifying one or more target concentrations in a biological sample using a detection apparatus (e.g., PCR apparatus 100). The blocks illustrated in FIG. 3 represent processes and/or methods that can be implemented and/or executed by one or more computing elements described below (e.g., by one or more computing devices). The computing elements can be standalone computing elements, networked computing elements, distributed computing elements, and/or embedded computing elements. For example, the computing elements can be integrated with PCR apparatus 100, an onsite standalone computing device, a cloud-based computing device, or a combination thereof.

Various processes of analysis pipeline 300 are performed to identify precise locations of each of thousands of partitions in a microfluidic array plate represented by an image of the partitions. Processes of analysis pipeline 300 are also performed to improve the accuracy of classifying the partition images using a machine-learning model. The training dataset of the machine-learning model is expanded by modifying existing training images, thereby increasing both the quantity and variety of the training images. An expanded training dataset can enhance the machine-learning model's capability of accurate classification. Processes of analysis pipeline 300 are further performed to increase signal-to-noise ratios of the detected fluorescence signals, apply spectral compensation to reduce crosstalk between channels, and perform thresholding to filter out false positive partition images and false negative partition images. Accordingly, by performing one or more processes of analysis pipeline 300, the accuracy of quantifying target concentrations is greatly improved.

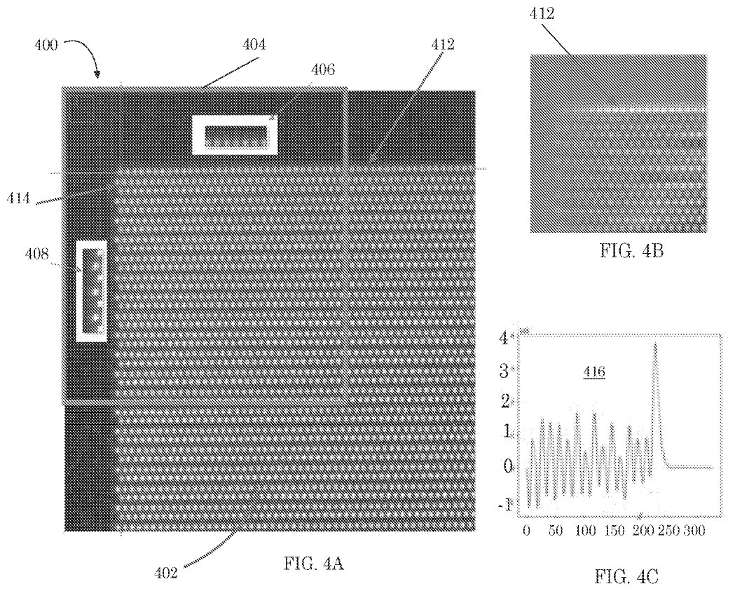

In some embodiments, analysis pipeline 300 begins with a process 302 for determining corner locations of an array of partitions. With reference to FIG. 4A, an image 400 represents an array of partitions 402. For illustration purposes, image 400 only shows a portion of array 402. Image 400 is post-PCR fluorescence image representing array 402 after the amplification of one or more targets in a biological sample is performed by thermal cycling. Each bright spot shown in image 400 thus represents a partition of array 402 after amplification. It is understood that the brightness of the spots in image 400 shown in FIG. 4A are for illustration purposes and can vary in any manner. For example, some partitions in array 402 may appear to be less bright or dark (e.g., because the partitions do not include a target). In some embodiments, image 400 is associated with a particular fluorescence channel (e.g., a channel using an ROX dye) of an PCR apparatus having multiplexing capabilities. While the below description uses a post-PCR image as an example, it is understood that a pre-PCR image can also be used to determine corner locations of an array of partitions.

In process 302, a computing device uses image 400 to determine locations associated with corners of array 402. Corner locations of an array of partitions are used subsequently for determining expected locations of partitions in array 402. Observed locations are then determined based on the expected locations and are used to obtain images of individual partitions. Next, the images of individual partitions are classified, the results of which are used for calculating target concentrations. The partition location determination process and the classification process are described in more detail below. The corner location determination process 302 is performed before many other processes in analysis pipeline 300.

With reference to FIG. 4A, array 402 is a two-dimensional array having four corners. A corner location determination process 302 may begin with any of the four corners. FIG. 4A uses the top left corner of array 402 as an illustration. To determine the location of the top left corner of array 402, a corner area 404 is first selected to indicate that the top left corner is under analysis. Corner area 404 may be annotated using, for example, a square or a rectangle shaped box having a predetermined dimension.

In one embodiment, dimensions of corner area 404 are predetermined such that corner area 404 is not too small or too large. An overly small corner area may not include enough partitions for correlating with template images to correctly find an edge. For example, as shown in FIG. 4A, a template image 406 represents seven partitions disposed along a horizontal edge of a template array of partitions. Correspondingly, the dimensions of corner area 404 may be predetermined such that 20-40 partitions disposed along a horizontal edge are included in corner area 404 for analysis. Template image 406 is described below in more detail.

An overly large corner area may also affect the correlation using template images due to, for example, warpage of the image of the array of partitions. Warpage of the image may cause poor correlation between template images with the image of the array of partitions, making it difficult to find edges of the array of partitions. Thus, a properly dimensioned corner area should include sufficient number of partitions based on the template images but also not be overly large to cause correlation difficulty. It is understood that proper dimensions of a corner area can be predetermined, by a computing device and/or by a user input, based on the dimensions of the array of partitions and the dimensions of the template images.

In one embodiment shown in FIG. 4A, corner area 404 is annotated and displayed. The annotation may be, for example, a colored box overlaying an image of the array of partitions to indicate the selected corner area. In FIG. 4A, corner area 404 is annotated by a colored box (e.g., a green box) overlying image 400 to indicate that the top left corner is selected to be analyzed.

As described above, template images are used for determining corner locations. A template image includes several portions that can be used to find an edge of an array. FIG. 4A illustrates a template image 406 used to correlate with a top edge 412 of array 402 and a template image 408 used to correlate with a left edge 414 of array 402. Template image 406 includes two portions. A first portion of template image 406 comprises several bright spots representing a plurality of partitions that are predetermined to be partitions disposed along a top edge. These predetermined partitions of template image 406 form, for example, a line pattern as shown in FIG. 4A. A second portion of template image 406 represents an area predetermined to have no partitions. In template image 406, this second portion is immediately adjacent to the first portion and is dark. Therefore, template image 406 is an image representing a known pattern of partitions disposed along a known top edge.

FIG. 4A further illustrates another template image 408, which is an image representing a known pattern of partitions disposed along a known left edge. In FIG. 4A, template image 408 also includes two portions. A first portion of template image 408 comprises several bright spots representing a plurality of partitions that are predetermined to be partitions disposed along a left edge. These predetermined partitions form, for example, a staggered pattern as shown in FIG. 4A. The staggered pattern may also be viewed as comprising two staggered lines. A part of the partitions represented in template image 408 form a first vertical line and another part of the partition form a second vertical line. The first and second vertical lines are offset from each other in the horizontal direction. For example, as shown in FIG. 4A, the partitions forming the first line may be disposed more towards the left than the partition forming the second line. And the partitions of the first line and the second line are staggered in alternating rows.

Like template image 406, template image 408 also has a second portion that represents an area predetermined to have no partitions. In FIG. 4A, this portion is shown as a dark area immediately adjacent to the first portion with the partitions forming the staggered line pattern. Unlike template image 406, the second portion of which is oriented in a horizontal direction, the second portion of template image 408 is oriented in a vertical direction. The two portions of the template image 408 thus represent a known pattern of partitions disposed along a known left edge. While FIG. 4A illustrates template images used for the top edge and the left edge, it is understood that similar template images can be configured and obtained for the bottom edge and the right edge.

A corner of a two-dimensional array is formed by two edges. For instance, in FIG. 4A, the top left corner of array 402 is formed by top edge 412 and left edge 414. As such, if the locations of the two edges are determined, the location of the corresponding corner can be determined. In one embodiment, two template images are used to determine locations of two edges, and in turn, a corner location formed by the two edges. For example, in FIG. 4A, template image 406 and template image 408 are obtained and used for determining locations of top edge 412 and left edge 414, respectively, that form the top left corner. Using template image 406 as an example, to determine the location of top edge 412, template image 406 is moved and/or rotated to correlate with corner area 404 to find top edge 412. If at least a part of the template image 406 is matched with top edge 412, the location of top edge 412 may be determined. For instance, by moving and/or rotating template image 406, if at least four or five out of the total seven partitions shown in template image 406 match with at least four or five partitions shown in corner area 404 of image 400, and if the dark portion shown in template image 406 also matches with the dark portion above top edge 412 in corner area 404, then top edge 412 is found. In some embodiments, if an initial match is found, template image 406 is further moved along a horizontal direction to see if additional or continued matches can be found. If so, there is high probability that top edge 412 is found. Once top edge 412 is found, its location can be readily determined by, for example, measuring the number of pixels from top edge 412 to the top edge of image 400.

In a similar manner, to determine the location of left edge 414 of array 402, template image 408 is moved and/or rotated to correlate with left edge 414. If at least a part of template image 408 is matched with left edge 414, the location of left edge 414 may be determined. For instance, by moving and/or rotating template image 408, if at least four or five out of the total seven partitions shown in template image 408 match with four or five partitions in corner area 404 and if the dark portion shown in template image 408 also matches with the dark portion to the left of left edge 414 in corner area 404, then left edge 414 is found. In some embodiments, if an initial match is found, template image 408 is further moved along a vertical direction to see if additional or continued matches can be found. If so, there is high probability that left edge 414 is found. Once left edge 414 is found, its location can be readily determined by, for example, measuring the number of pixels from left edge 414 to the left edge of image 400.

When a match between a template image and an edge is found, visual indications may be displayed on image 400 and in other manners. FIGS. 4B and 4C illustrate such exemplary visual indications using the top edge as an example. In FIG. 4B, the brightness of certain partitions disposed along top edge 412 is increased to indicate a likely match between template image 406 and top edge 412. FIG. 4C illustrates another type of visual indication of a match, which is a signal peak having a height that is significantly greater than the others.

With reference to FIG. 4A, after the location of top edge 412 and the location of left edge 414 are determined, the location of the top left corner of array 402 can be determined. In one embodiment, the location of the top left corner is represented by horizontal and vertical coordinates in number of pixels from the left and top edges, respectively. The location of the top left corner of array 402 shown in FIG. 4A may thus be represented by a coordinate pair such as (135, 133). While FIG. 4A uses the top left corner as an example, it is understood that locations of other corners of array 402 can be determined in a similar manner. Moreover, when additional images of the same array 402 or different arrays are obtained, for example, from different fluorescence channels, corner locations of the same array or different arrays can be similarly determined using the above-described techniques.

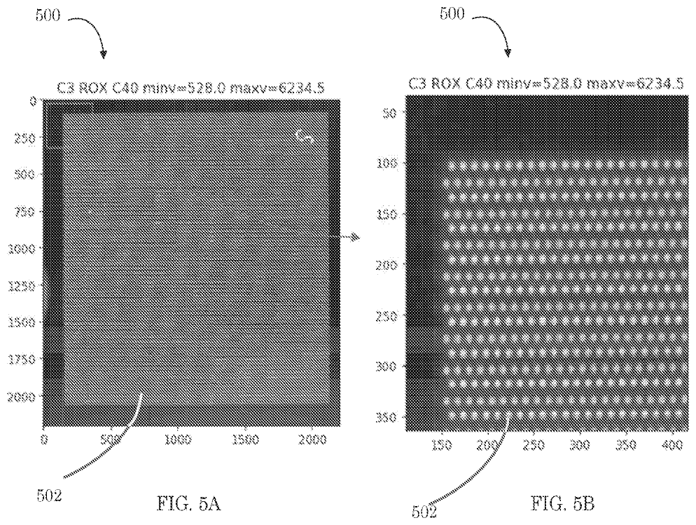

With reference back to FIG. 3, the corner locations determined in process 302 are provided to process 304, in which individual partition locations in an array of partitions are determined. As described above, an array of partitions may be arranged in multiple rows and columns. FIG. 5A illustrates such an array 502 represented in image 500. FIG. 5B shows a zoom-in image representing the top left corner area of array 502. As illustrated in FIGS. 5A and 5B, array 502 includes many partitions (e.g., about 20,000) arranged in rows and columns. These partitions are illustrated by the bright spots in FIGS. 5A and 5B. Ideally, these partitions are equally spaced from one another in the underlying microfluidic array plate, such that the partitions form a regular repeating pattern. Further, the images of the individual partitions would also ideally show that the partitions are arranged in rows and columns with equal spacing. If the partitions themselves and the images of them are both ideal, then locations of the partitions can be readily determined from the corner locations of the array of partitions by a simple calculation.

However, many factors may affect the partitions in a microfluidic array plate and the images of the partitions, rendering deviations from the ideal arrangement of the partitions and/or the ideal image thereof. For example, the microfluidic array plate and/or some microchambers of partitions may have defects such that the spacings between some partitions may be smaller or greater than a designed or desired spacing. In addition, contamination of the partitions and/or the microfluidic array plate (e.g., dust, fiber, surface contamination) may affect the images of the partitions, such that they are not a precise representation of the partition arrangement. The imaging system may introduce errors as well. As an example, when capturing the images of the partitions, the camera position may have some error, so the microfluidic array plate and/or the partitions may not be always centered in the image. The microfluidic array plate and/or the partitions may have some rotations with respect to the camera such that the rows and columns of the partitions are not perfectly horizontal and vertical in the image. Also, the camera lenses may introduce some distortion such that the rows and columns of the partitions are not perfectly straight, but rather they may have some small curvatures that are dependent on locations in the image.

The above listed factors are just some examples that may affect the precise determination of locations of the partitions represented in an image. Many other factors may also affect the images of the partitions and in turn affect an accurate determination of the partition locations. If partition locations are not determined accurately, the classifications of individual partition images to either positive or non-positive may also be inaccurate. Inaccurate classifications may in turn lead to errors in quantifying target concentrations, and thus negatively impact the accuracy of the PCR measurement. Thus, it is desirable to accurately determine the locations of partitions as represented by the image of the partitions.

With reference to FIGS. 5A and 5B, partitions in array 502 are arranged in rows and columns. Therefore, array 502 has four corners. The locations of the four corners of array 502 can be determined using techniques described above. The locations of the corners can be represented in number of pixels in a pair of X and Y coordinates (i.e., number of pixels measured from the relevant edges in the horizontal and vertical directions). For example, the location of the top left corner of array 502 may be represented as a coordinate pair measured in pixels such as (135, 132), where “135” is the distance of the top left corner from the left edge of image 500 in numbers of pixels and “132” is the distance of the top left corner from the top edge of image 500 in numbers of pixels. The locations of the top right corner, the bottom left corner, and the bottom right corner of array 502 may be similarly represented as (2073, 127), (130, 2049), and (2085, 2044), respectively. In the above examples, the location of top left corner of image 500 is set to be the base coordinate point (0, 0), and the locations of the corners are measured with respect to the base coordinate point. It is understood that any other base coordinate point or system can be used.

After corner locations of an array of partitions are determined, expected locations of partitions in the array can be calculated. Expected locations of partitions are locations calculated according to design and/or manufacturing specifications of the microfluidic array plate. For example, the design and/or manufacturing specifications may provide a row spacing between any two immediately neighboring partitions in a same row. Similarly, a column spacing between any two immediately neighboring rows may also be provided by the specifications. Further, in some embodiments, two immediately neighboring rows of partitions may be offset in the horizontal direction and the offset is also known according to the design and/or manufacturing specifications. An example of such an offset is illustrated in FIG. 5B, where two immediately neighboring rows may be offset by about the width of a partition.

Using one or more of the known row spacing, column spacing, and offset values provided by the design and/or manufacturing specifications, the expected locations of the partitions can be calculated based on the determined corner locations. An expected location is also referred to as an expected center location of a partition. For example, a coordinate pair (x, y) of the expected center location of a partition may be used to represent the expected location of the partition. In some embodiments, the expected locations of partitions can be represented by expected row center locations and corresponding expected column center locations. An expected row center location is the expected location of a partition center in the horizontal direction (i.e., the X direction). An expected row center location of a partition can be calculated based on locations of two corners (e.g., the top left and top right corners) and the known row spacing between two immediately neighboring partitions in a same row. For instance, if the top left corner has an X-axis location of 10 pixels and the row spacing is 9 pixels, all the expected row center location of partitions in the first row are determined (e.g., 19 pixels, 28 pixels, 37 pixels, and so on). An expected column center location is the expected location of a partition center in the vertical direction (i.e., the Y direction). Similar to the expected row center locations, expected column center locations of the partitions can be calculated based on the locations of two corners (e.g., the top left and bottom left corners) and the known column spacing between two immediately neighboring rows. In some embodiments, the offset between two immediately neighboring rows is also taken into account when calculating the expected row center or column center partition locations.

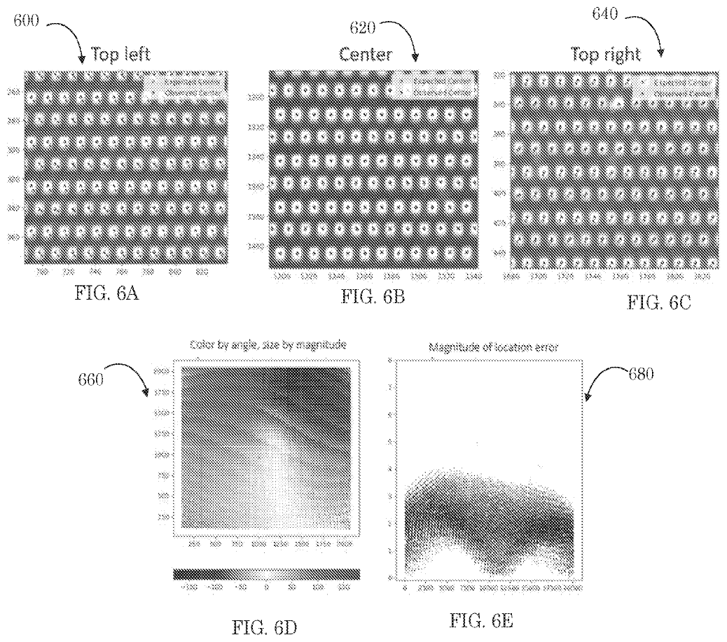

As described above, many factors may affect the partitions and images thereof. Therefore, locations of the partitions observed using an image of the array of partitions may or may not be the same as the expected locations. FIGS. 6A-6C illustrate images showing deviations of observed locations of the partitions from the expected locations. An observed location of a partition is also referred to as an observed center location of the partition. In one embodiment, a pair of (x, y) coordinates of the observed center location of a partition is used to represent the observed location of the partition.

FIG. 6A illustrates an image 600 of partitions located near the top left portion of an array of partitions. In image 600, the observed center locations of at least some partitions do not overlap with the expected center locations of the same partitions. Instead, the observed center locations of those partitions deviate from the expected center locations by a small distance toward, for example, the top left direction.

FIG. 6B illustrates an image 620 of partitions located near the center portion of the array. In image 620, the observed center locations of at least some partitions largely overlap with the expected center locations of the same partitions. FIG. 6C illustrates an image 640 of partitions located near the top right portion of the array. In image 640, the observed center locations of at least some partitions do not overlap with the expected center locations of the same partitions. Instead, the observed center locations of those partitions deviate from the expected center locations by a small distance toward, for example, the top right direction. The deviations of the observed center locations of partitions from their expected center locations may be caused by many factors as described above (e.g., image warpage). Due to the deviations, the expected center locations cannot be used directly as the locations for obtaining individual partition images. Instead, it is more desirable to use the observed center locations. It is understood that FIGS. 6A-6C illustrate only some examples of deviations. Observed locations of partitions may deviate from expected locations in any manner and are not limited to those shown in FIGS. 6A-6C.

FIG. 6D illustrates an error map 660 visually displaying the deviations of the observed locations from the expected locations and the directions of the deviations. In FIG. 6D, the center portion of error map 660 is largely white colored, indicating that the observed locations and the expected locations in the center portion of the array largely overlap. The left portion of error map 660 in FIG. 6D is largely red colored, indicating the observed locations in the left portion of the array deviate from the expected locations toward certain direction(s) (e.g., the top left direction). And the right portion of error map 660 in FIG. 6D is largely blue colored, indicating the observed locations deviate from the expected locations toward another direction(s) (e.g., the top right direction).

In some embodiments, color intensity can be used to represent the degree of deviation or the error magnitude. The greater the color intensity, the larger the deviations or error magnitudes. In error map 660, for example, the color intensity becomes greater toward the top left and top right corners, indicating larger deviations or error magnitudes in those area.

FIG. 6E illustrates a diagram 680 showing the magnitudes of location errors with respect to the number of partitions in the array of partitions, providing another way to visualize the location errors. It is understood that the location errors and their magnitudes can be visualized and displayed in any desired manner other than those illustrated in FIGS. 6D and 6E (e.g., using different colors in the error map, using a bar chart, scatter plots, etc.).

Because the observed locations of the partitions may deviate from the expected locations, it is desirable to determine the observed locations of the partitions so that images of individual partitions can be obtained based on the observed locations. With reference to FIG. 6A, if an image of a particular partition is obtained using the expected locations (indicated by a red dot), the partition image may not represent the entire observed partition. Rather, the image may capture unwanted dark area between the observed partitions while missing part of the observed partition, causing loss of detected signals. Images obtained based on expected partition locations may subsequently be falsely classified (e.g., as a non-positive partition because a part of the image is dark).

Therefore, the expected locations of the partitions may not be directly used to obtain images of individual partitions. Instead, an expected location is used as an initial prediction to determine the observed location. In some embodiments, match filtering can be applied to images of the partitions at or near the expected locations. Match filtering correlates a predetermined template partition image with an image under analysis. A match filter can be, for example, a two-dimensional filter configured for image matching.

In some embodiments, one or more template partition images are obtained. The template partition images are two-dimensional images representing predetermined partitions. To determine an observed location of a particular partition, one or more of these template partition images are moved horizontally, vertically, and/or rotated at or near the expected location of the partition. Based on such movement and/or rotation of the template partition images, the template partition images are correlated with the image of a particular partition by matching at least a part of the template partition images with the image of the partition. After a match is found, a signal is generated to indicate the match (e.g., an increase of brightness of a particular partition image, or a signal indicating a peak).

In some embodiments, after a match is found, the partition location can be determined. For example, the partition location can be calculated based on a movement distance and/or rotation angles of the template partition images from a base coordinate point of the image of array of partitions. In some embodiments, the partition location obtained by match filtering can be represented as a coordinate pair including a correlated row center location and a correlated column center location, collectively referred to as a correlated center location or correlated location of the partition. Similar to the expected location (also referred to as expected center location), the correlated location can also be represented by a pair of (X, Y) coordinates in number of pixels.

The correlated location of a partition obtained by match filtering may or may not be an observed location. As illustrated in FIGS. 6A-6C, an observed location often deviates from an expected location of the same partition by a small distance, e.g., by several pixels. In some embodiments, if the correlated location of a particular partition obtained based on match filtering is more than a threshold distance from an expected location of the same partition, then the correlated location may not be the observed location of the partition. Therefore, to determine whether a correlated location is an observed location, the correlated location of a particular partition obtained based on match filtering is compared to the expected location of the same partition. For example, a correlated row center location and an expected row center location can be compared to determine if they are within a predetermined row error threshold. Similar, a correlated column center location and an expected column center location can be compared to determine if they are within a predetermined column error threshold.

In some embodiments, the correlated location is determined to be the observed location if the correlated row center location and the expected row center location are within the predetermined row error threshold and if the correlated column center location and the expected column center location are within a predetermined column error threshold. If one or both conditions are not met, the correlated location may not be the observed location.

In some embodiments, a correlated location and an expected center location are used to calculate a distance between them. The distance is then compared to a threshold distance. If the distance is within a threshold distance, then the correlated location is determined to be the observed location, and vice versa.

In some embodiments, match filtering may not correctly identify a correlated location of a particular partition. For example, the partition may have design or manufacturing defects. There may be contamination of the partition or reflection from the surface of a microfluidic array plate. Other factors described above may also affect the identification a partition by the match filtering. As a result, the correlated location may not be the observed location even if the correlated location is within a threshold distance of the expected location. For example, a reflective artifact in the vicinity of an expected location may cause the match filter to falsely identify it as genuine partition image. But in this case, the correlated location of the artifact is not an observed location of a partition. As another example, a microchamber of the partition may be incorrected filled with a PCR test sample, which may cause difficulty for the match filtering to correctly identify the partition.

In some embodiments, during or after the process of match filtering, a degree of correlation associated with determining a correlated row center location and determining a correlated column center location is generated. The degree of correlation may be in the form of a probability, goodness of matching, and/or a confidence level. Based on the degree of correlation, a numerical score or a pass/fail indication may be assigned to indicate how likely a correlated row center location and the corresponding correlated column center location represent an observed location of a partition. For example, even if a microchamber of a partition may be incorrected filled with a PCR test sample, the partition may still be assigned a passing indication or a passing score if the degree of correlation is nonetheless above a predetermined threshold. The degree of correlation may be determined by taking in account many factors such as the deviation between the correlated location and the expected location, the degree of matching between the template partition images and the image of a particular partition, the image quality and conditions, the signal and noise ratio, the orientation of the partition image, or the like.

In some embodiments, distances between partitions can also be used in determining how likely a correlated location is an observed location of a partition. As described above, by design, partitions are ideally positioned regularly in rows and columns. The distances between the immediately neighboring partitions are thus typically identical or nearly identical. This property of the distances between partition can be used to filter out correlated locations that are unlikely observed locations. In one embodiment, one or more distances between correlated locations are calculated. The distances so calculated can be compared with one another to determine whether a particular correlated center location is abnormal. For example, the regular distance between two immediately neighboring partitions may be about 10 pixels. A distance between two correlated locations can be calculated using correlated row center locations and/or a correlated column center locations of the two correlated locations. If the distance is more than a threshold distance (e.g., the threshold distance is 12 pixels, but the calculated distance is 20 pixels), the correlated locations of one or both these two partitions may not be observed locations. In other words, one or both correlated locations may be a result of false matching by the match filtering. The distance calculation can therefore be used as a post-match filtering step to remove false matching in determining observed locations.

With reference back to FIG. 3, in some embodiments, an optional background image subtraction process 306 can be performed to improve the quality of images of partitions before they are provided to a machine-learning model based classification process 308. While process 306 is illustrated in FIG. 3 as being performed after process 304 for determining observed locations of the partitions, it can also be performed in any step before classification process 308.

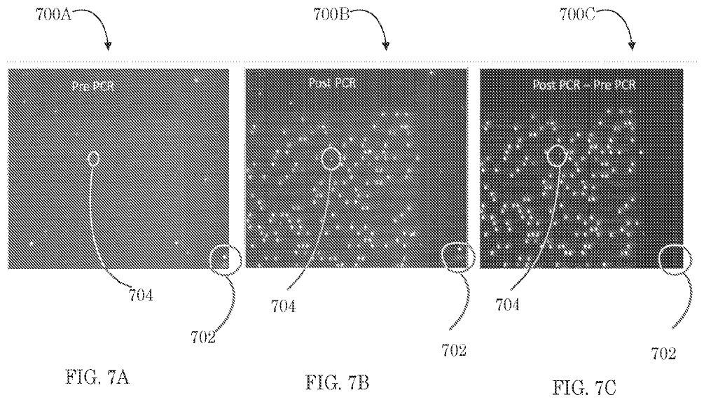

Process 306 is illustrated using FIGS. 7A-7C. In process 306, a pre-PCR image 700A and a post-PCR image 700B are obtained. Pre-PCR image 700A represents an array of partitions before amplification of one or more targets in a biological sample. Post-PCR image 700B represents the same array of partitions after amplification of the one or more targets. Images 700A and 700B show a portion of the array of partitions for illustration purposes. Using pre-PCR image 700A and post-PCR image 700B, image subtraction can be performed to remove or mitigate certain image artifacts to improve image quality. For example, pre-PCR image 700A has an artifact 702. The same artifact 702 also exists in post PCR-image 700B. This type of artifact is referred to as a correlated artifact. A correlated artifact can be removed or mitigated by performing image subtraction. In some embodiments, image subtraction can be performed by aligning the pre-PCR image and the post-PCR image at the pixel level and then performing pixel-by-pixel subtraction. For example, FIG. 7C shows a result image 700C after performing subtraction. Image 700C no longer has the correlated artifact 702, thereby improving the image quality.

An image artifact represents an undesirable image defect. Image subtraction can be performed to mitigate or remove an image artifact associated with image defects in both the pre-PCR image and the post-PCR image. For example, dust on a camera lens may cause an image defect on all pre- and post-PCR images. Artifacts caused by such image defects are readily removed or mitigated by performing image subtraction. In some embodiments, image subtraction can be used to mitigate or remove an image artifact associated with contamination represented in both the pre-PCR image and the post-PCR image. For example, a particle or another contamination in a particular partition may cause an image defect on all pre- and post-PCR images. Artifacts caused by such contamination can also be readily removed or mitigated by performing image subtraction.

FIGS. 7A-7C also illustrate an uncorrelated artifact 704. As shown in FIG. 7B, post-PCR image 700B has an artifact 704. But artifact 704 does not exist in pre-PCR image 700A shown in FIG. 7A. This type of artifact is referred to as an uncorrelated artifact. An uncorrelated artifact may not be removed or mitigated by image subtraction. An uncorrelated artifact may be removed or ignored using match filtering as described above or using a machine-learning model based classification process 308 as described in more detail below.

With reference back to FIG. 3, using the observed locations of partitions determined in process 304, images of individual partitions can be obtained (e.g., by image cropping at or near the observed locations or reproducing using the original image of the array of partitions). These images of individual partitions are provided to a classification process 308, with or without the optional background image subtraction process 306. In some embodiments, classification process 308 is a machine-learning model based classification process that can identify positive partition images and non-positive partition images.

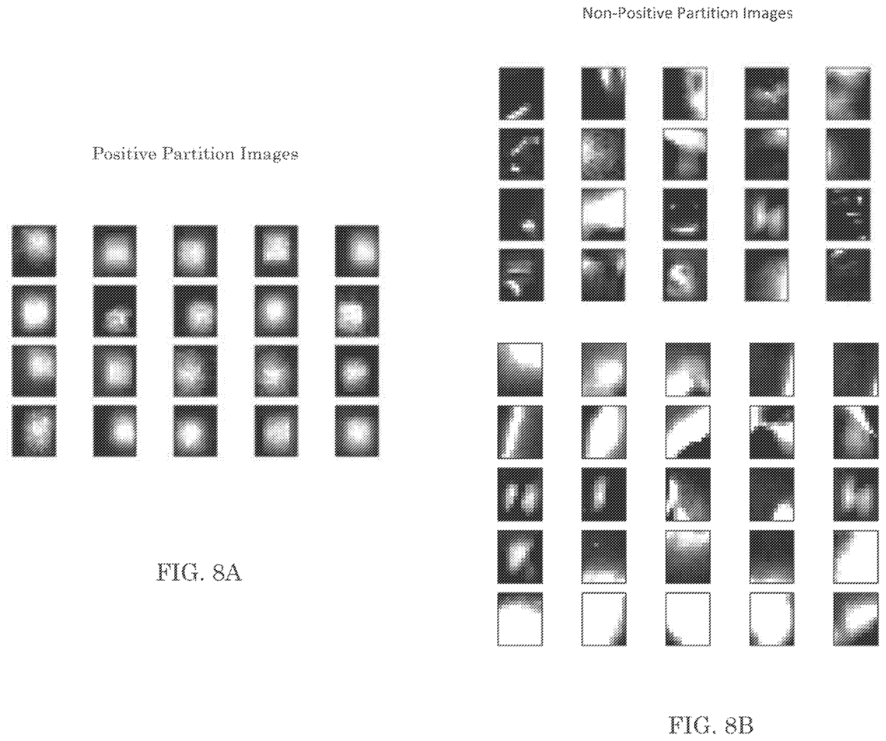

FIG. 8A illustrates examples of positive partition images. Positive partition images represent positive partitions, which correspond to an existence of one or more target concentrations in the partitions. A positive partition includes a target (e.g., a DNA molecule) that has been amplified by thermal cycling so that it is detectable via fluorescence signals. A positive partition thus includes a large number of target PCR amplicons of the original DNA. The positive partition images are subsequently used for quantifying the target PCR amplicon concentration in a biological sample.

FIG. 8B illustrates examples of non-positive partition images. Non-positive partition images include negative partition images, images associated with defective microchambers of partitions, images associated with contaminated partitions, images associated with defective fillings of microchambers of partitions, defective images of partitions, and any post-PCR partition images other than positive partition images. A negative partition image corresponds to a non-existence of a target presence in a partition. If a particular partition does not have a target (e.g., a DNA target molecule such as a PCR amplicon), then thermal cycling in PCR did not produce target amplicons. As a result, a fluorescence image of the negative partition appears mostly or completely dark due to the lack of fluorescence signals. Some of these images are shown in FIG. 8B. These images may be classified as non-positive partition images.

Images associated with defective partitions are also examples of non-positive partition images. For example, a defective partition can include design and/or manufacturing defects in a microchamber of the partition, defects in microfluidic array plate such as a reflective surface of the plate, a defective filling of a microchamber of the partition, and any other defects of a partition or the microfluidic array plate. Such defective partitions may or may not include a target (e.g., a DNA target molecule such as nucleic acids originally present in the biological sample or PCR amplicons generated from such nucleic acids). While thermal cycling in PCR may produce target amplicons of a target, defective partitions may affect the fluorescence signal detection and image generation. Images generated based on defective partitions may not be used to accurately quantify the target concentration in the biological sample under analysis. Some of these images are shown in FIG. 8B. These images may be classified as non-positive partition images.

Images associated with contaminated partitions are also examples of non-positive partition images. Partitions may be contaminated with, for example, dusts, fibers, particles, parasite DNAs, or the like. Contaminated partitions may or may not include a target. While thermal cycling in PCR may produce amplicons of a target, contaminated partitions may affect the fluorescence signal detection and image generation. Images generated based on contaminated partitions may not be used to accurately quantify the target concentration in the biological sample under analysis. Some of these images are shown in FIG. 8B. These images may also be classified as non-positive partition images.

Images themselves can also be defective. Defective images may also be classified as non-positive partition images. Imaging defects can include, for example, defects associated with the imaging system such as dust on a camera lens, a corrupted image, image distortion, or the like. These images may not be used to accurately quantify the target concentration in the biological sample under analysis. Some of these images are shown in FIG. 8B. These images may also be classified as non-positive partition images.

While negative partition images, images of defective partitions, images of contaminated partitions, and defective images are described above as examples of non-positive partition images, it is understood that any other partition images that are not positive partition images may also be classified as non-positive partition images.

A machine-learning model can use images that are pre-identified as positive partition images and non-positive partition images for training the model. For example, both the positive partition images and non-positive partition images in FIGS. 8A and 8B can be used as training images for training the machine-learning model.

Positive partition images and non-positive partition images can be obtained from past PCR tests. For example, a group of post-PCR images can be classified and annotated (manually or automatically) as positive partition images and non-positive partition images. In some embodiments, training of a machine-learned model may need many annotated images to provide enough quantity and enough variety of both positive and non-positive partition images. Therefore, images obtained only from past PCR analyses may or may not be sufficient for training a machine-learning model.

If images obtained from past PCR analyses are insufficient, additional training images may be necessary. To obtain additional images for training a machine-learning model, in some embodiments, a set of non-positive partition images can be generated based on another set of non-positive partition images. For example, a first set of images is obtained from past PCR tests. The images in the first set are pre-identified and annotated as non-positive partition images. The images in the first set can be modified to obtain a second set of images. Such image modification can include, but not limited to, one or more of rotating, editing, cropping, distorting, mirroring, brightening, darkening, changing a contrast of, changing a color of, and changing a pattern of, the first set of images. The modified images in the second set can also be pre-identified and annotated as non-positive partition images. The second set can thus be included in the group of non-positive partition images used for training the machine-learning model, thereby increasing the quantity and variety of the available training images.

In a similar manner, in some embodiments, a set of non-positive partition images can be generated based on a set of positive partition images. For example, a third set of images is obtained from past PCR tests. The images in the third set are pre-identified and annotated as positive partition images. The positive partition images in the third set can be modified to obtain a fourth set of images, which can be non-positive partition images. Such image modification can include, but not limited to, one or more of rotating, editing, cropping, distorting, mirroring, brightening, darkening, changing a contrast of, changing a color of, and changing a pattern of, the first set. The modified images in the fourth set can be pre-identified and annotated as non-positive partition images. The fourth set of images can thus be included in the group of non-positive partition images used for training the machine-learning model, thereby increasing the quantity and variety of the available training images.

In the above examples, a set of non-positive partition images and/or a set of positive partition images are modified and used to generate another set of non-positive partition images. In a similar manner, a set of non-positive partition images and/or a set of positive partition images can be modified and used to generate another set of positive partition images. In some embodiments, the non-positive partition images and/or positive partition images being modified are also referred to as seed images. By modifying seed images, many other images can be generated to expand the quantity of the training images. Modification of the seed images can also be configured to generate different types of non-positive partition images and/or positive partition images, thereby greatly improving the variety of the training images for the machine-learning model.

In some embodiments, a set of positive partition images and a set of non-positive partition images are combined to generate one or more datasets for training the machine-learning model. The datasets may include a training dataset, a validation dataset, and a testing dataset. For example, ⅓ of the images are used as a training dataset; ⅓ of the images are used as a validation dataset; and ⅓ of the images are used as a testing dataset. The datasets may include image files and/or features extracted from the image files.

Using the one or more datasets, one or more computing devices (e.g., a server) iteratively train the machine-learning model to determine a set of parameters. The set of parameters can include, for examples, weights and features of the machine-learning model. The training can be performed using, for example, a stochastic gradient descent method and/or its extensions and variants such as the Adaptive Moment Estimation (Adam) method, implicit updates method (ISGD), the momentum method, the averaged stochastic gradient descent method, the Adaptive Gradient (AdaGrad) method, and the root mean square propagation (RMSProp) method.

According to various embodiments, the machine-learning model used for classification of images can be, for example, a convolutional neural network (CNN). A CNN includes an input layer, one or more hidden layers, and an output layer. The hidden layers include layers that perform convolutions such as one or more convolutional layers. A CNN may also include local and/or global pooling layers along with the convolutional layers. The pooling layers are for reducing the dimensions of data. While CNN is used as an example of the machine-learning model for classification of images, it is understood that extensions and variants of CNN and/or other types of neural networks for image classification may also be used.

After training, the trained machine-learning model is configured with the set of parameters determined from the training. The trained machine-learning model can then be used for classifying images to positive partition images or non-positive partition images. As described above, observed locations of partitions represented in a post-PCR image are determined. Based on the observed locations, images of individual partitions can be obtained (e.g., by image cropping or reproducing at or near the observed locations). These individual partition images are then provided to the trained machine-learning model. Optionally, before the partition images are provided to trained machine-learning model, image subtraction is performed to improve the post-PCR image quality. The trained machine-learning model then classifies each individual partition images as a positive partition image or a non-positive partition image. In some embodiments, the trained machine-learning model determines a probability that a particular partition image is a positive partition image. Based on the probability, the image is classified as a positive or non-positive partition image.

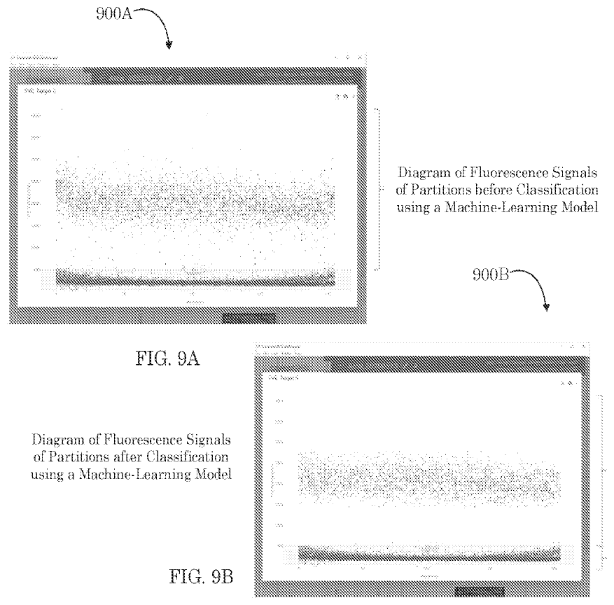

A trained machine-learning model can greatly improve the accuracy of classification of positive versus non-positive partition images. FIGS. 9A-9B illustrate such an improvement. In both FIGS. 9A and 9B, the horizontal axis represents the partition numbers (e.g., total of 20,000 partitions) and the vertical axis represents the fluorescence signal intensity. FIG. 9A shows a diagram 900A of fluorescence signals of partitions before the classification using a machine-learning model is performed. In diagram 900A, the fluorescence signals are plotted according to the detected signals. As described above, many of the fluorescence signals representing positive partitions may be false positives due to a variety of factors such as partition defects, image artifacts, contaminations, or the like. FIG. 9B illustrates a diagram 900B of fluorescence signals of partitions after the classification using a machine-learning model is performed. Using a trained machine-learning model, many of the false positive signals can be removed or filtered out by correctly classifying the false positive partition images as non-positive partition images. Accordingly, the machine-learning model based classification process 308 can significantly improve the accuracy of determining positive partition images.

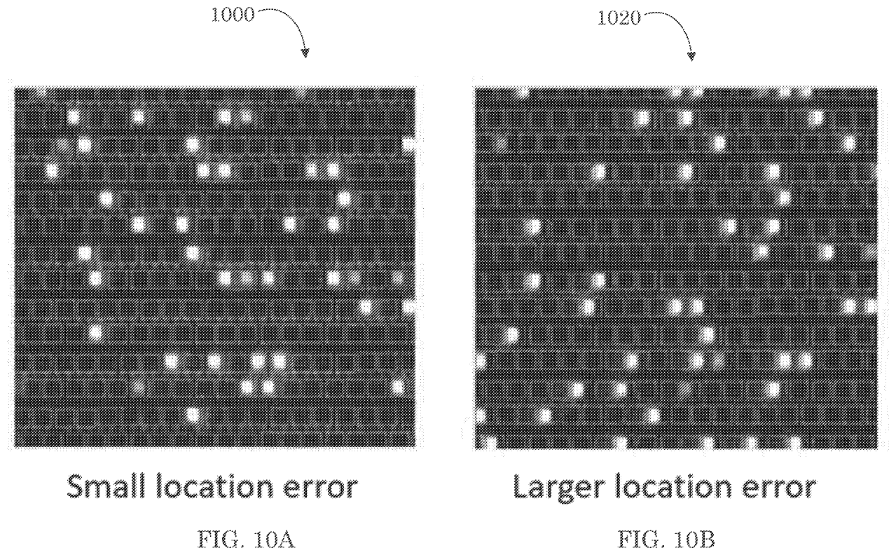

With reference back to FIG. 3, in process 310, fluorescence signals represented in the partition images are summarized. While FIG. 3 illustrates that process 310 is performed after process 308, process 310 may also be performed before process 308. For example, the fluorescence signals represented in the partition images can be summarized before the partition images are classified. In some embodiments, the dimension of a partition is about 9 pixels by 11 pixels. Accordingly, a 9×11 grid or frame is applied at each observed location of a partition. An observed location of a particular partition may have error due to various factors. A location error refers to the difference between the observed location and an actual center location of a partition. FIG. 10A illustrates an image 1000 representing partitions with small location errors; while FIG. 10B illustrates an image 1020 representing partitions with large location errors. In FIGS. 10A and 10B, the 9×11 grids are shown as red rectangular boxes applied at each of the observed locations of the partitions. Due to location errors, however, the boxes may or may not completely overlap with the partition images. Such location errors may lead to an inaccurate summary of the signals in a partition. In general, the greater the location error, the less the summary accuracy and resolution.

In some embodiments, to summarize the fluorescence signals represented in a partition image of a particular partition, a 9×11 grid or frame is applied at the observed location of the partition. Next, several brightest pixels (e.g., three) in the corresponding 9×11 grid are ignored. Fluorescence signals of the next group of brightest pixels (e.g., 30 pixels) are then averaged to obtain the summarized value of the signals in a particular partition represented by the partition image. In some embodiments, several darkest pixels are also ignored in calculating the summarized value of the signals. Fluorescence signals in a partition represented by a non-positive partition (e.g., a negative partition) image may also be summarized in a similar manner or in a different manner (e.g., simply taking the average of all the pixels). It is understood that other method of summarizing signals of a partition may also be used. For instance, summarizing signals may include, but not limited to, integrating, summing, averaging, weighted averaging, etc. of the signals.

With reference back to FIG. 3, in some embodiments, after fluorescence signals represented in a partition image are summarized for all desired partition images (e.g., all positive partition images and all negative partition images), a spectral compensation process 312 is performed. As described above, a PCR apparatus may have multiplexing capabilities by using multiple fluorescence channels. The PCR apparatus with multiplexing capabilities is able to simultaneously detect and analyze different fluorescence signals having different spectra (showing different colors). However, the different fluorescence signals may have overlapping spectra such that one channel may pick up signals from a dye measured by another channel. This is often referred to as crosstalk between the channels. Crosstalk may degrade the signal integrity and may result inaccuracy in measuring and interpreting the signals. To mitigate or remove crosstalk between the channels, spectrum compensation can be applied.

Spectrum compensation may be applied for each summary of fluorescence signals represented in a corresponding partition image. In some embodiments, spectrum compensation is based on linear spectral unmixing techniques that analyze the spectral mixing and estimate a set of pure spectral signatures (often referred to as endmembers) and fractions of these endmembers (often referred to as abundances). One of such linear unmixing techniques uses a linear mixing model (LMM), which assumes that the spectrum of a mixed pixel is a linear combination of the pure spectra of the components present in that pixel weighted by their fractional coverage. Based on the linear spectral unmixing results, spectrum compensation values can be determined to remove or mitigate crosstalk between channels.

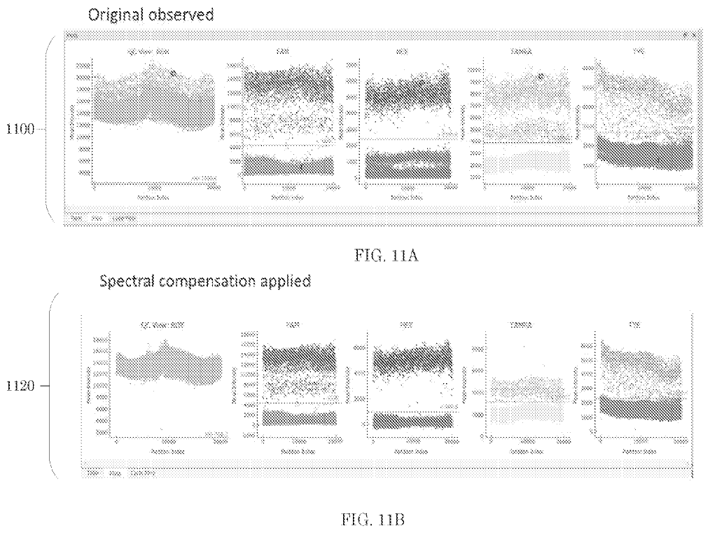

In some embodiments, spectrum compensation values are generated during a PCR apparatus calibration run and then saved in a dye registry file stored in the PCR apparatus. Each PCR test using the PCR apparatus can then retrieve the spectrum compensation values and apply the spectrum compensation using the values. Thus, spectrum compensation can be applied on a per PCR apparatus basis. FIG. 11A illustrates diagrams 1100 of detected different-colored fluorescence signals in five different fluorescence channels with crosstalk, i.e., before spectral compensation is applied. FIG. 11B illustrates diagrams 1120 of the fluorescence signals after spectral compensation is applied. Each diagram in FIGS. 11A and 11B has a horizontal axis representing the partition numbers (e.g., total of 20,000 partitions) and the vertical axis representing the fluorescence signal intensity. In the diagrams 1120 shown in FIG. 11B, many signals from crosstalk between the channels have been filtered out, resulting in a more accurate representation of the detected fluorescence signals in each channel.

With reference back to FIG. 3, in some embodiments, after the process 312 of spectral compensation, a partition table can be generated and displayed to a user. The partition table can include various data including, for example, a location of each partition (positive and/or negative), a summary of signals represented in each partition image, index of the partitions, the fluorescence channel used for a particular image, or the like. The partition table thus represents a dataset that can be used for various purposes, such as visualizing the signals, calculating target presence or concentrations, analyzing the PCR tests, or the like.

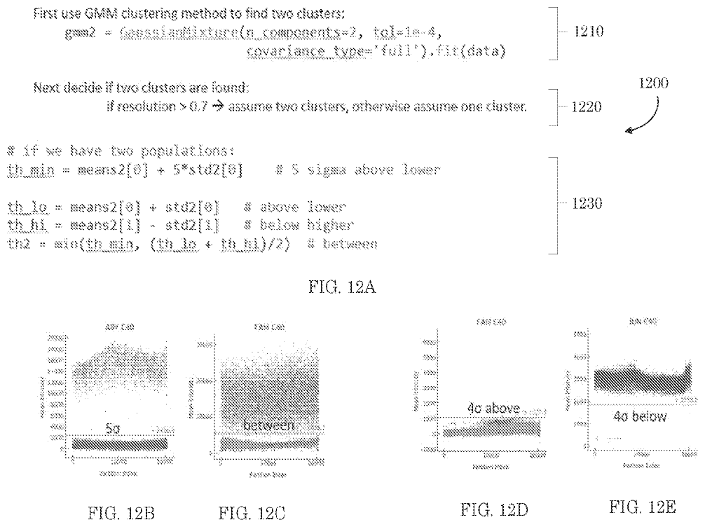

With reference still to FIG. 3, in some embodiments, one or more results obtained from one or more previously described processes (e.g., processes 308, 310, 312, and/or 314) may be further processed before target presence is detected or target concentrations are quantified. For instance, in process 316, the summaries of signals represented in each partition image can be further processed, based on which one or more partition images may be rejected. FIG. 12A illustrates an exemplary process 1200 for processing the summaries of signals using a mixing model such as a Gaussian Mixture Model (GMM).

A mixture model can be applied to the populations of summaries of signals. A mixture model is used to make statistical inferences about the properties of sub-populations of data given only observations on the pooled population, without sub-population identity information. Sub-populations are also referred to as clusters. A GMM is one type of mixture model. A GMM can be a non-Bayesian GMM or a Bayesian GMM. As shown in FIG. 12, in step 1210 of process 1200, a GMM clustering method is applied to the population of the signal summaries. A GMM clustering method assumes that there are a certain number of Gaussian distributions, and each of these distributions represent a cluster. Hence, a GMM tends to group the data points belonging to a single distribution together, thereby forming one or more clusters. It is understood that other clustering methods (e.g., a k-means, k-medoids, and expectation-maximization algorithm, a density-based spatial clustering of applications with noise, i.e., DBSCAN algorithm, an ordering points to identify the clustering structure, i.e., OPTICS algorithm, hierarchical clustering algorithms, etc.) may also be used to form one or more clusters.

In step 1220 of process 1200, the number of clusters is determined. In some embodiments, the number of clusters is determined based on the shape, scale, and/or desired clustering resolution (e.g., 0.7). For example, each of FIGS. 12B and 12C illustrates a plot showing two clusters of summarized fluorescence signals; while each of FIGS. 12D and 12E illustrates a plot showing one cluster of summarized fluorescence signals. An optimal number of clusters can provide a balance between maximum compression of the data using a single cluster, and maximum accuracy by assigning each data point to its own cluster. Various methods can be used to determine the number of clusters, including the elbow method, the X-means clustering method, the information criterion approach, the silhouette method, the cross-validation method, etc.

In step 1230, based on the determined number of clusters, various maximum and minimum signal summary thresholds for rejecting one or more partition images can be configured. For example, partition images having summaries of signals greater than the maximum signal summary threshold are rejected and not used in detecting target presence or detecting target presence or quantifying target concentration. In some embodiments, if a signal summary of a partition image is greater than a maximum threshold, the partition image may be associated with a partition that has defects, contamination (e.g., dust), or the like. Therefore, these type of partition images are rejected. The minimum signal summary threshold can also be configured to reject partition images having summaries of signals less than the minimum signal summary threshold. In some embodiments, if a signal summary of a partition image is less than a minimum threshold, the partition image may be associated with a partition that is incorrectly filled or unfilled with PCR test sample. These types of partition images are also rejected.

Various maximum and minimum signal summary thresholds can be configured based on the GMM clustering results and/or based on user input. As shown in FIG. 12A, in some embodiments, a minimum signal summary threshold is configured based on the low or high values of the signal summaries and standard deviations (e.g., one sigma or five sigma). FIGS. 12B and 12C visually display such thresholds when two clusters are determined. FIGS. 12D and 12E illustrate that one cluster is determined and an exemplary threshold configured in such a case. If one cluster is determined, the machine-learning model based classification process 308 can be used to determine whether a summarized signal corresponds to a positive partition image or a negative partition image, as described above.

With reference back to FIG. 3, a mixing model (e.g., a GMM) can also be used in process 318, which further processes one or more results obtained from one or more previously described processes (e.g., processes 308, 310, 312, and/or 314). For example, based on the mixing model, a signal summary threshold is configured to reject partition images having a signal summary value above the threshold. A partition image classified by the machine-learning based classification process as a positive partition image may be a false positive partition image. But using the signal summary threshold, the false positive partition images are rejected and not used as positive partition images for detecting target presence or detecting target presence or quantifying target concentration.

In some embodiments, process 318 is also performed to identify statistically improbable situations such as a number n of consecutive position partitions. Positive partitions are typically generated by a random process for disposing the PCR test samples to the array of partitions. For example, as shown in FIG. 1, some partitions in the array of partitions 120 contain a target analyte, e.g., a nucleotide sequence, but some don't. This process is often random. Therefore, statistically, it is rare or improbable that a large number of consecutive partitions can all be positive partitions. In some embodiments, such a large number of consecutive positive partitions can be predicted using the Goulden-Jackson Cluster Method and/or approximated using the Feller Success Run prediction. For each image representing an array of partitions, these methods or similar ones can be applied to identify a large number of consecutive partitions, which are statistically improbable. The partition images corresponding to these partitions are subsequently rejected and not used for detecting target presence or quantifying target concentration.

With reference still to FIG. 3, in process 320, target presence is detected or concentrations are quantified. In some embodiments, to detect target presence or quantify the target concentrations, the number of total partitions, the number of positive partition images (less the ones rejected in the previous various processes), and the total number of accepted partition images (including both positive and negative less the ones rejected in the previous various processes) are obtained. Using Poisson statistics, the target presence can be detected or concentrations can be quantified within a defined confidence interval. For example, the number of targets in the original solution may be correlated to the number of samples producing a positive detection signal represented by the positive partition images. By performing one or more of the above-described processes (e.g., machine-learning model based classification, observed location determination, spectral compensation, partition rejection by thresholding, rejecting long consecutive positive partitions), the target concentration can be quantified in a more accurate manner than traditional analog-type PCR apparatuses.

With reference to FIG. 3, in process 322, visual reports and charts of the results of target detection or concentration quantification can be generated and displayed to a user.

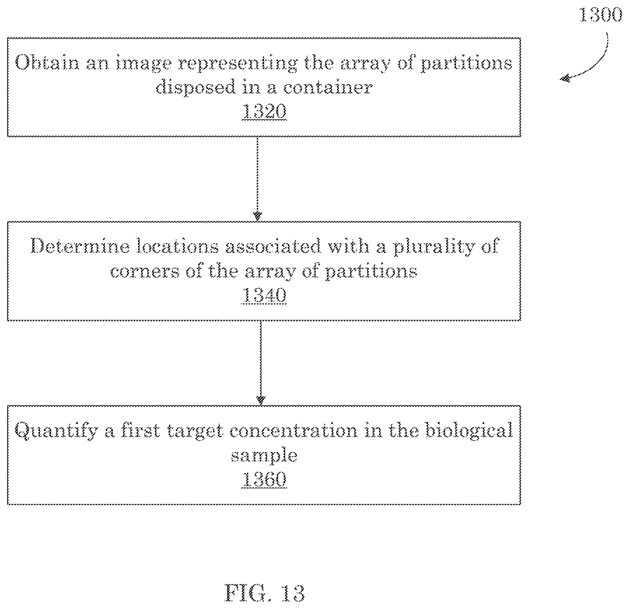

FIGS. 13-16 illustrate various methods for detecting target presence or quantifying target concentration or for training a machine-learning model accordingly to various embodiments. With reference to FIG. 13, a method 1300 for quantifying target concentration is provided. Method 1300 begins with a step 1320, during which an image representing an array of partitions disposed in a container is obtained. Image 400 shown in FIG. 4A is one example of such an image. FIG. 1 illustrates an exemplary container that includes a microfluidic array plate 110. In one embodiment, the image includes a pre-PCR image representing the array of partitions before amplification of one or more targets in a biological sample. In one embodiment, the image includes a post-PCR image representing the array of partitions after amplification of one or more targets in the biological sample. Further, the image can be associated with one of a plurality of fluorescence channel of a PCR apparatus. In some embodiments, the plurality of fluorescence channels is associated with different spectral wavelengths.

Method 1300 further includes a step 1340, during which locations associated with a plurality of corners of the array of partitions are determined. In some embodiments, the location determination includes selecting a first corner area using the image obtained in step 1320, obtaining a first template image and a second template image; and determining a location of the first corner based on the first template image and the second template image. The first corner area includes a first corner formed by a first edge of the array and a second edge of the array of partitions. FIG. 4A illustrates such a corner area 404.

In one embodiment, selecting the first corner area includes obtaining dimensions of an area to-be-selected; selecting the first corner area based on the dimensions of the area to-be-selected; and displaying an annotation of the first corner area. The annotation overlays the image representing the array of partitions. As shown in FIG. 4A, a green box overlays image 400 as an exemplary annotation of corner area 404.

In one embodiment, determining the location of the first corner (e.g., the top left corner of array 402 in FIG. 4A) includes determining a location of the first edge (e.g., edge 412 in FIG. 4A) using the first template image (e.g., template image 406); determining a location of the second edge (e.g., edge 414 in FIG. 4A) using the second template image (e.g., template image 408); and determining the location of the first corner based on the location of the first edge and the location of the second edge. In determining the location of the first edge (e.g., edge 412 in FIG. 4A) using the first template image, the first template image (e.g., template image 406 in FIG. 4A) is moved and/or rotated. Based on one or both of moving and rotating the first template image, the first template image is correlated with the first corner area to find the first edge by matching at least a part of the first template image with the first edge. The location of the first edge is then determined based on a result of correlating the first template image with the first edge.

The location of the second edge (e.g., edge 414 in FIG. 4A) can be similarly determined. In one embodiment, the second template image (e.g., template image 408 in FIG. 4A) is moved and/or rotated. Based on one or both of moving and rotating the second template image, the second template image is correlated with the first corner area to find the second edge by matching at least a part of the second template image with the second edge. The location of the second edge is determined based on a result of correlating the second template image with the second edge.

In some embodiments, the first template image (e.g., template image 406 in FIG. 4A) includes a first portion and a second portion. The first portion represents a plurality of predetermined partitions forming a first pattern. The second portion represents an area predetermined to have no partitions. The second portion is immediately adjacent to the first portion. In one embodiment, the first pattern includes a single line pattern formed by the plurality of predetermined partitions.

Similarly, the second template image (e.g., template image 408) includes a third portion and a fourth portion. The third portion represents a plurality of predetermined partitions forming a second pattern. The fourth portion represents an area predetermined to have no partitions. The fourth portion is immediately adjacent to the third portion. In one embodiment, the second pattern includes a first line pattern and a second line pattern. The first line pattern is formed by a part of the predetermined partitions. The second line pattern is formed by another part of the predetermined partitions. The first line pattern and the second line pattern are offset from each other.

It is understood that locations of additional corners can be determined in a similar manner in step 1340. For example, using the image (e.g., image 400 in FIG. 4A) representing the array of partitions (e.g., array 402), one or more additional corner areas including one or more corresponding additional corners are selected. One or more additional template images (e.g., a template image corresponding to the bottom edge and right edge of array 402) are obtained. Based on two or more of the first template image, the second template image, and the one or more additional template images, one or more locations of the one or more additional corners are determined.

With reference back to FIG. 13, in step 1360, based on the locations associated with the plurality of corners, a first target concentration in the biological sample can be quantified. In one embodiment, quantifying the first target concentration in the biological sample includes determining partition locations of the array of partitions based on the locations associated with the plurality of corners (e.g., by performing process 304 in FIG. 3); classifying each partition image representing a partition of the array of partitions as a positive partition image or a non-positive partition image (e.g., by performing process 308 in FIG. 3; and quantifying the first target concentration in the biological sample based on a classification result of at least some of the partition images (e.g., by performing one or more processes 310, 312, 314, 316, 318, and 320).

Additional images of the same array or different arrays of partitions can be processed according to method 1300. For example, in a similar manner as described above, one or more additional images associated with one or more fluorescence channels (e.g., a channel using a ROX based dye) of an PCR apparatus are obtained. Based on the one or more additional images, additional locations associated with the plurality of corners of the array of partitions are determined. Based on the additional locations, one or more additional target concentrations in the biological sample are quantified.

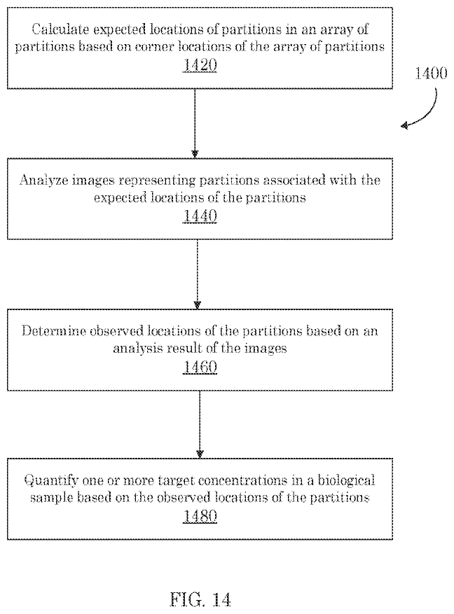

With reference to FIG. 14, a method 1400 for quantifying one or more target concentrations in a biological sample using an analyte detection apparatus is provided. The analyte detection apparatus (e.g., PCR apparatus 100 in FIG. 1) is configured to analyze an array of partitions of the biological sample. Method 1400 begins with a step 1420, during which expected locations of partitions (e.g., those expected locations shown in FIGS. 6A-6C) in the array of partitions are calculated based on corner locations of the array of partitions. In one embodiment, prior to calculating the expected locations, corner locations of the array of partitions are obtained using an image representing the array of partitions (e.g., image 400 in FIG. 4A).

In some embodiments, calculating the expected locations includes calculating expected row center locations of the partitions based on a predetermined row spacing between two immediately neighboring partitions and calculating expected column center locations of the partitions based on a predetermined column spacing between two immediately neighboring rows.

In step 1440, images representing partitions associated with the expected locations of the partitions are analyzed. In some embodiments, analyzing the images includes performing one or both of moving and rotating one or more template partition images. Based on one or both of moving and rotating the one or more template partition images, the one or more template partition images are correlated with the partition images by matching at least a part of the one or more template partition images with the partition images associated with the expected locations of the partitions. In one embodiment, the one or more template partition images include images representing predetermined partitions.

In step 1460, observed locations of the partitions (e.g., those observed locations shown in FIGS. 6A-6C) are determined based on an analysis result of the images. In one embodiment, determining the observed locations includes determining correlated row center locations of the partitions based on the analysis result of the images and determining correlated column center locations of the partitions based on the analysis result of the images. Further, it is determined whether the correlated row center locations and corresponding expected row center locations are within a predetermined row error threshold. Similarly, it is determined whether the correlated column center locations and corresponding expected column center locations are within a predetermined column error threshold.

In one embodiment, in accordance with a determination that at least one correlated row center location and the corresponding at least one expected row center location are within the predetermined row error threshold and a determination that at least one correlated column center location and the corresponding at least one expected column center location are within the predetermined column error threshold, a score is calculated. The score indicates a probability that the at least one correlated row center location and the corresponding at least one correlated column center location correspond to at least one observed location.

Based on the score, at least one of the observed locations is determined. In one embodiment, calculating the score includes estimating a degree of correlation associated with determining the at least one correlated row center location and determining the at least one correlated column center location. A score is then assigned based on the degree of correlation. The score indicates whether the at least one correlated row center location and the corresponding at least one correlated column center location correspond to at least one of the observed locations.

In some embodiments, step 1460 further includes determining one or more distances between the partitions using the correlated row center locations and correlated column center locations. Based on the one or more distances, it is determined whether at least one correlated row center location and at least one corresponding correlated column center location do not correspond to at least one of the observed locations.

In step 1480, one or more target concentrations in the biological sample are quantified based on the observed locations of the partitions. In one embodiment, quantifying the target concentration includes providing images representing partitions associated with the observed locations of the partitions to a trained machine-learning model. Using the trained machine-learning model, the images are classified as positive partition images or non-positive partition images. Based on a classification result of the classification of at least some of the partition images, the one or more target concentrations in the biological sample are quantified.

In one embodiment, method 1400 further includes one or more steps for image subtraction illustrated in FIGS. 7A-7C. For example, image subtraction includes obtaining a pre-PCR image representing the array of partitions before amplification of one or more targets in the biological sample and obtaining a post-PCR image representing the array of partitions after amplification of the one or more targets in the biological sample. Image subtraction is then performed using the pre-PCR image and the post-PCR image. The image subtraction can be performed by one or more of mitigating or removing an artifact associated with image defects in both the pre-PCR image and the post-PCR image; and mitigating or removing an artifact associated with contamination represented in both the pre-PCR image and the post-PCR image.

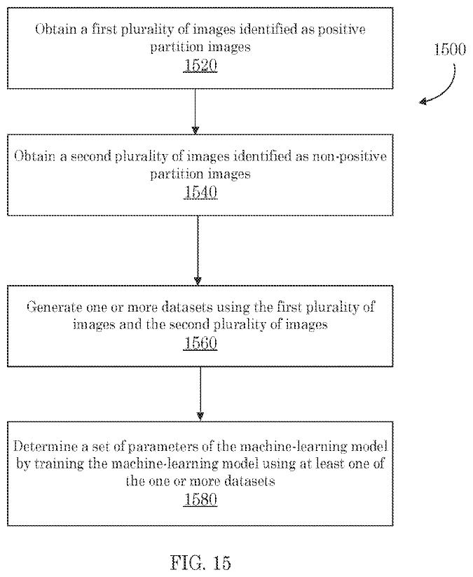

With reference to FIG. 15, a method 1500 is provided. Method 1500 is for training a machine-learning model used for analyzing one or more biological samples by an analyte detection apparatus. Method 1500 is performed by one or more computing devices.

In some embodiments, method 1500 begins with step 1520, during which a first plurality of images (e.g., images shown in FIG. 8A) identified as positive partition images is obtained. The positive partition images are associated with positive partitions, which correspond to an existence of one or more target concentrations in the positive partitions.

In step 1540, a second plurality of images (e.g., images shown in FIG. 8B) identified as non-positive partition images is obtained. The second plurality of images includes one or more images modified from one or more other images identified as non-positive partition images. The non-positive partition images include at least one negative partition image associated with a negative partition, which corresponds to a non-existence of a target concentration in the negative partition. The non-positive partition images may also include at least one image associated with a defective microchamber of a partition. The non-positive partition images may also include at least one image associated with a contaminated partition. The non-positive partition images may also include at least one image associated with a defective filling of a microchamber of a partition. The non-positive partition images may also include at least one defective image of a partition.

In some embodiments, obtaining the second plurality of images (step 1540) includes obtaining a first subset of the second plurality of images identified as non-positive partition images. The first subset of the second plurality of images is modified to obtain a second subset of the second plurality of images, which are identified as non-positive partition images. Step 1540 also includes including the second subset in the second plurality of images. The modifying of the first subset of the second plurality of images includes one or more of rotating, editing, cropping, distorting, mirroring, brightening, darkening, changing a contrast of, changing a color of, and changing a pattern of, the first subset of the second plurality of images.

In some embodiments, obtaining the second plurality of images (step 1540) includes obtaining a first subset of the first plurality of images identified as positive partition images. The first subset of the first plurality of images is modified to obtain a third subset of the second plurality of images, which are identified as non-positive partition images. Step 1540 also includes including the third subset in the second plurality of images. The modifying of the first subset of the first plurality of images includes one or more of rotating, editing, cropping, distorting, mirroring, brightening, darkening, changing a contrast of, changing a color of, and changing a pattern of, the first subset of the first plurality of images.

In step 1560, one or more datasets are generated using the first plurality of images (e.g., images shown in FIG. 8A) and the second plurality of images (e.g., image shown in FIG. 8B). In one embodiment, the one or more datasets includes a training dataset, a validation dataset, and a testing dataset.

In step 1580, a set of parameters of the machine-learning model is determined. The set of parameters is determined by iteratively training the machine-learning model using the one or more datasets. Based on a result of the iterative training, the set of parameters of the machine-learning model is determined. In one embodiment, the machine-learning model includes a convolutional neural network (CNN).

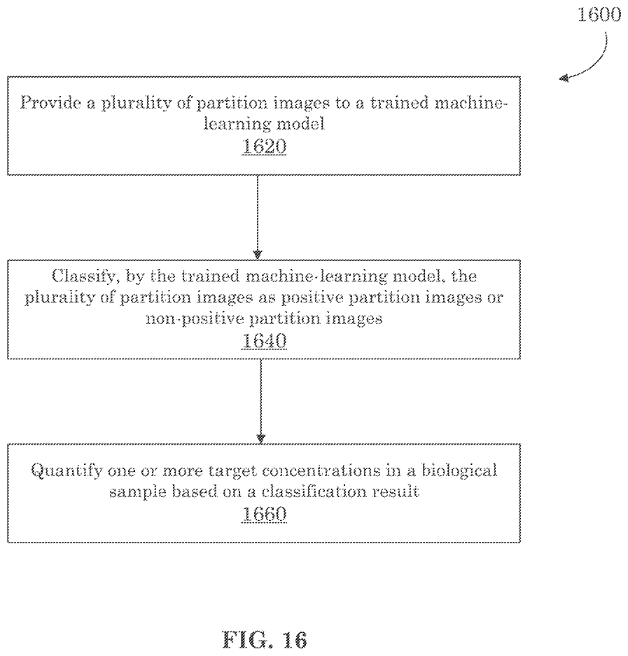

With reference to FIG. 16, a method 1600 is provided. Method 1600 is for quantifying one or more target concentrations in a biological sample using an analyte detection apparatus (e.g., PCR apparatus 100 in FIG. 1) configured to analyze an array of partitions of the biological sample. In one embodiment, method 1600 begins with a step 1620, during which a plurality of partition images is provided to a trained machine-learning model. The plurality of partition images represents a corresponding plurality of partitions that are at least a subset of the array of partitions. Method 1600 may include other steps before step 1620. For example, method 1600 may include determining a plurality of partition locations of the plurality of partitions based on an image (e.g., image 400 in FIG. 4A) representing the array of partitions of the biological sample. Method 1600 may also include obtaining the plurality of partition images based on the corresponding plurality of partition locations.

In some embodiments, method 1600 may further include other steps performed prior to determining the plurality of partition locations of the plurality of partitions. These steps may include steps for performing image subtraction. Image subtraction includes obtaining a pre-PCR image representing the plurality of partitions before amplification of one or more targets in the biological sample and obtaining a post-PCR image representing the plurality of partitions after amplification of the one or more targets in the biological sample. Image subtraction is then performed using the pre-PCR image and the post-PCR image to obtain the plurality of partition images provided to the trained machine-learning model. In one embodiment, image subtraction includes one or more of mitigating or removing an artifact associated with image defects in both the pre-PCR image and the post-PCR image; and mitigating or removing an artifact associated with contamination represented in both the pre-PCR image and the post-PCR image.

In step 1640, the plurality of partition images is classified by the trained machine-learning model as positive partition images or non-positive partition images. The trained machine-learning model is trained by using one or more images modified from one or more other images identified as non-positive partition images. In one embodiment, classifying the plurality of partition images includes, for each partition image of the plurality of partition images, determining a probability that the partition image is a positive partition image. Based on the probability, the partition image is classified as a positive partition image or a non-positive partition image.

In step 1660, the one or more target concentrations in the biological sample are quantified based on a classification result. In some embodiments, quantifying the target concentrations includes processing the classification result based on a threshold of positive partition images and quantifying the one or more target concentrations in the biological sample based on the processed classification result.

Systems, apparatus, and methods described herein may be implemented using digital circuitry, or using one or more computers using well-known computer processors, memory units, storage devices, computer software, and other components. Typically, a computer includes a processor for executing instructions and one or more memories for storing instructions and data. A computer may also include, or be coupled to, one or more mass storage devices, such as one or more magnetic disks, internal hard disks and removable disks, magneto-optical disks, optical disks, etc.

Systems, apparatus, and methods described herein may be implemented using computers operating in a client-server relationship. Typically, in such a system, the client computers are located remotely from the server computers and interact via a network. The client-server relationship may be defined and controlled by computer programs running on the respective client and server computers. Examples of client computers can include desktop computers, workstations, portable computers, cellular smartphones, tablets, or other types of computing devices.

Systems, apparatus, and methods described herein may be implemented using a computer program product tangibly embodied in an information carrier, e.g., in a non-transitory machine-readable storage device, for execution by a programmable processor; and the method processes and steps described herein, including one or more of the steps of FIGS. 3-16, may be implemented using one or more computer programs that are executable by such a processor. A computer program is a set of computer program instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

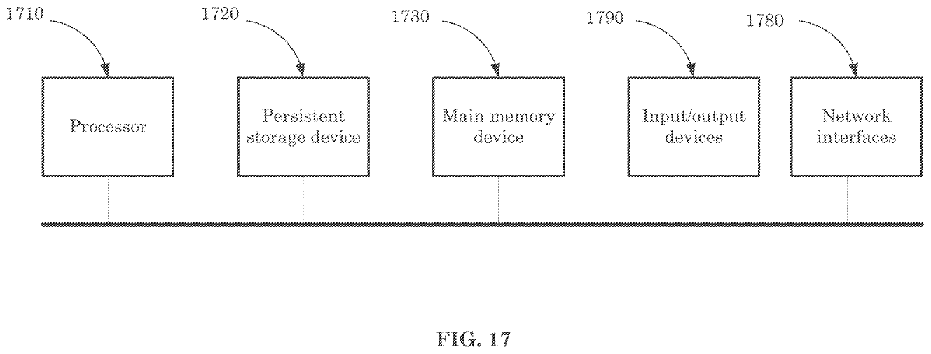

A high-level block diagram of an exemplary apparatus that may be used to implement systems, apparatus and methods described herein is illustrated in FIG. 17. Apparatus 1700 comprises a processor 1710 operatively coupled to a persistent storage device 1720 and a main memory device 1730. Processor 1710 controls the overall operation of apparatus 1700 by executing computer program instructions that define such operations. The computer program instructions may be stored in persistent storage device 1720, or other computer-readable medium, and loaded into main memory device 1730 when execution of the computer program instructions is desired. For example, processor 1710 may comprise one or more components of PCR apparatus 100. Thus, the method steps of FIGS. 3-16 can be defined by the computer program instructions stored in main memory device 1730 and/or persistent storage device 1720 and controlled by processor 1710 executing the computer program instructions. For example, the computer program instructions can be implemented as computer executable code programmed by one skilled in the art to perform an algorithm defined by the method steps of FIGS. 3-16. Accordingly, by executing the computer program instructions, the processor 1710 executes an algorithm defined by the method steps of FIGS. 3-16. Apparatus 1700 also includes one or more network interfaces 1780 for communicating with other devices via a network. Apparatus 1700 may also include one or more input/output devices 1790 that enable user interaction with apparatus 1700 (e.g., display, keyboard, mouse, speakers, buttons, etc.).

Processor 1710 may include both general and special purpose microprocessors and may be the sole processor or one of multiple processors of apparatus 1700. Processor 1710 may comprise one or more central processing units (CPUs), and one or more graphics processing units (GPUs), which, for example, may work separately from and/or multi-task with one or more CPUs to accelerate processing, e.g., for various image processing applications described herein. Processor 1710, persistent storage device 1720, and/or main memory device 1730 may include, be supplemented by, or incorporated in, one or more application-specific integrated circuits (ASICs) and/or one or more field programmable gate arrays (FPGAs).

Persistent storage device 1720 and main memory device 1730 each comprise a tangible non-transitory computer readable storage medium. Persistent storage device 1720, and main memory device 1730, may each include high-speed random access memory, such as dynamic random access memory (DRAM), static random access memory (SRAM), double data rate synchronous dynamic random access memory (DDR RAM), or other random access solid state memory devices, and may include non-volatile memory, such as one or more magnetic disk storage devices such as internal hard disks and removable disks, magneto-optical disk storage devices, optical disk storage devices, flash memory devices, semiconductor memory devices, such as erasable programmable read-only memory (EPROM), electrically erasable programmable read-only memory (EEPROM), compact disc read-only memory (CD-ROM), digital versatile disc read-only memory (DVD-ROM) disks, or other non-volatile solid state storage devices.

Input/output devices 1790 may include peripherals, such as a printer, scanner, display screen, etc. For example, input/output devices 1790 may include a display device such as a cathode ray tube (CRT), plasma or liquid crystal display (LCD) monitor for displaying information to a user, a keyboard, and a pointing device such as a mouse or a trackball by which the user can provide input to apparatus 1700.

Any or all of the functions of the systems and apparatuses discussed herein may be performed by processor 1710, and/or incorporated in, an apparatus such as PCR apparatus 100. Further, PCR apparatus 100 and/or apparatus 1700 may utilize one or more neural networks or other deep-learning techniques performed by processor 1710 or other systems or apparatuses discussed herein.

One skilled in the art will recognize that an implementation of an actual computer or computer system may have other structures and may contain other components as well, and that FIG. 17 is a high-level representation of some of the components of such a computer for illustrative purposes.

The foregoing specification is to be understood as being in every respect illustrative and exemplary, but not restrictive, and the scope of the invention disclosed herein is not to be determined from the specification, but rather from the claims as interpreted according to the full breadth permitted by the patent laws. It is to be understood that the embodiments shown and described herein are only illustrative of the principles of the present invention and that various modifications may be implemented by those skilled in the art without departing from the scope and spirit of the invention. Those skilled in the art could implement various other feature combinations without departing from the scope and spirit of the invention.

What is claimed is:

1. A method for detecting presence of a target in a biological sample, comprising:
obtaining an image representing an array of partitions disposed in a container;
determining, based on the image representing the array of partitions, locations associated with a plurality of corners of the array of partitions; and
quantifying, based on the locations associated with the plurality of corners, a first target concentration in the biological sample.

2. The method of claim 1, wherein the image representing the array of partitions comprises a pre-PCR image representing the array of partitions before amplification of one or more targets in the biological sample.

3. The method of claim 1, wherein the image representing the array of partitions comprises a post-PCR image representing the array of partitions after amplification of one or more targets in the biological sample.

4. The method of any of claims 1-3, wherein determining the locations associated with the plurality of corners of the array of partitions comprises:
selecting, using the image representing the array of partitions, a first corner area comprising a first corner formed by a first edge of the array and a second edge of the array;
obtaining a first template image and a second template image; and
determining, based on the first template image and the second template image, a location of the first corner.

5. The method of claim 4, wherein selecting the first corner area comprises:
obtaining dimensions of an area to-be-selected;
selecting the first corner area based on the dimensions of the area to-be-selected; and
displaying an annotation of the first corner area, the annotation overlaying the image representing the array of partitions.

6. The method of claim 4, wherein determining the location of the first corner comprises:
determining a location of the first edge using the first template image;
determining a location of the second edge using the second template image; and
determining the location of the first corner based on the location of the first edge and the location of the second edge.

7. The method of claim 6, wherein determining the location of the first edge using the first template image comprises:
performing one or both of moving and rotating the first template image;
correlating, based on one or both of moving and rotating the first template image, the first template image with the first corner area to find the first edge by matching at least a part of the first template image with the first edge; and
determining the location of the first edge based on a result of correlating the first template image with the first edge.

8. The method of claim 6, wherein determining the location of the second edge using the second template image comprises:
performing one or both of moving and rotating the second template image;
correlating, based on one or both of moving and rotating the second template image, the second template image with the first corner area to find the second edge by matching at least a part of the second template image with the second edge; and
determining the location of the second edge based on a result of correlating the second template image with the second edge.

9. The method of any of claims claim 4-8, wherein:
the first template image comprises a first portion and a second portion;
the first portion represents a plurality of predetermined partitions forming a first pattern; and
the second portion represents an area predetermined to have no partitions, the second portion being immediately adjacent to the first portion.

10. The method of claim 9, wherein the first pattern comprises a single line pattern formed by the plurality of predetermined partitions.

11. The method of any of claims 4-10, wherein the second template image comprises a third portion and a fourth portion, wherein:
the third portion represents a plurality of predetermined partitions forming a second pattern; and
the fourth portion represents an area predetermined to have no partitions, the fourth portion being immediately adjacent to the third portion.

12. The method of claim 11, wherein the second pattern comprises a first line pattern and a second line pattern, the first line pattern being formed by a part of the predetermined partitions, and the second line pattern being formed by another part of the predetermined partitions, the first line pattern and the second line pattern being offset from each other.

13. The method of any of claims 4-12, further comprising:
selecting, using the image representing the array of partitions, one or more additional corner areas comprising one or more corresponding additional corners;
obtaining one or more additional template images; and
determining, based on two or more of the first template image, the second template image, and the one or more additional template images, one or more locations of the one or more additional corners.

14. The method of any of claims 1-13, wherein the image is associated with a first fluorescence channel of a PCR apparatus comprising a plurality of fluorescence channels including the first fluorescence channel, the plurality of fluorescence channels being associated with different spectral wavelengths.

15. The method of claim 14, further comprising:
obtaining one or more additional images, the one or more additional images being associated with one or more fluorescence channels of the PCR apparatus;
determining, based on the one or more additional images, additional locations associated with the plurality of corners of the array of partitions; and
quantifying, based on the additional locations, one or more additional target concentrations in the biological sample.

16. The method of claim 14, wherein one of the plurality of fluorescence channels comprises a channel using a 6-carboxy-X-rhodamine (ROX) based dye.

17. The method of any of claims 1-16, wherein quantifying the first target concentration in the biological sample comprises:
determining, based on the locations associated with the plurality of corners, partition locations of the array of partitions;
classifying each partition image representing a partition of the array of partitions as a positive partition image or a non-positive partition image; and
quantifying, based on a classification result of at least some of the partition images, the first target concentration in the biological sample.

18. The method of any of claims 1-17, wherein the container comprises a microfluidic array plate.

19. A non-transitory computer readable medium comprising a memory storing one or more instructions which, when executed by one or more processors of at least one computing device, perform quantification of one or more target concentrations in a biological sample using an analyte detection apparatus configured to analyze an array of partitions of the biological sample by processing comprising:
obtaining an image representing the array of partitions disposed in a container;
determining, based on the image representing the array of partitions, locations associated with a plurality of corners of the array of partitions; and
quantifying, based on the locations associated with the plurality of corners, a first target concentration in the biological sample.

20. A system for quantifying one or more target concentrations in a biological sample using an analyte detection apparatus configured to analyze an array of partitions of the biological sample, the system comprising:
one or more processors of at least one computing device; and
a memory storing one or more instructions, when executed by the one or more processors, cause the one or more processors to perform processing comprising:
obtaining an image representing the array of partitions disposed in a container;
determining, based on the image representing the array of partitions, locations associated with a plurality of corners of the array of partitions; and
quantifying, based on the locations associated with the plurality of corners, a first target concentration in the biological sample.

21. A method of quantifying one or more target concentrations in a biological sample using an analyte detection apparatus configured to analyze an array of partitions of the biological sample, the method comprising:
calculating expected locations of partitions in the array of partitions based on corner locations of the array of partitions;
analyzing images representing partitions associated with the expected locations of the partitions;
determining observed locations of the partitions based on an analysis result of the images; and
quantifying the one or more target concentrations in the biological sample based on the observed locations of the partitions.

22. The method of claim 21, further comprising obtaining corner locations of the array of partitions using an image representing the array of partitions.

23. The method of any of claims 21-22, wherein calculating the expected locations of the partitions in the array of partitions based on corner locations of the array of partitions comprises:
calculating expected row center locations of the partitions based on a predetermined row spacing between two immediately neighboring partitions; and
calculating expected column center locations of the partitions based on a predetermined column spacing between two immediately neighboring rows.

24. The method of any of claims 21-23, wherein analyzing the images representing partitions associated with the expected locations of the partitions comprises:
performing one or both of moving and rotating one or more template partition images; and
correlating, based on one or both of moving and rotating the one or more template partition images, the one or more template partition images with the partition images by matching at least a part of the one or more template partition images with the partition images associated with the expected locations of the partitions.

25. The method of claim 24, wherein the one or more template partition images comprise images representing predetermined partitions.

26. The method of any of claims 21-25, wherein determining the observed locations of the partitions based on the analysis result of the images comprises:
determining correlated row center locations of the partitions based on the analysis result of the images; and
determining correlated column center locations of the partitions based on the analysis result of the images.

27. The method of claim 26, further comprising:
determining whether the correlated row center locations and corresponding expected row center locations are within a predetermined row error threshold; and
determining whether the correlated column center locations and corresponding expected column center locations are within a predetermined column error threshold.

28. The method of claim 27, further comprising:
in accordance with a determination that at least one correlated row center location and the corresponding at least one expected row center location are within the predetermined row error threshold and a determination that at least one correlated column center location and the corresponding at least one expected column center location are within the predetermined column error threshold,
calculating a score indicating a probability that the at least one correlated row center location and the corresponding at least one correlated column center location correspond to at least one observed location; and
determining, based on the score, at least one of the observed locations.

29. The method of claim 28, wherein calculating the score comprises:
estimating a degree of correlation associated with determining the at least one correlated row center location and determining the at least one correlated column center location; and
assigning, based on the degree of correlation, a score indicating whether the at least one correlated row center location and the corresponding at least one correlated column center location correspond to at least one of the observed locations.

30. The method of claim 26, further comprising:
determining one or more distances between the partitions using the correlated row center locations and correlated column center locations; and
determining, based on the one or more distances, whether at least one correlated row center location and at least one corresponding correlated column center location do not correspond to at least one of the observed locations.

31. The method of any of claims 21-30, further comprising,
obtaining a pre-PCR image representing the array of partitions before amplification of one or more targets in the biological sample;
obtaining a post-PCR image representing the array of partitions after amplification of the one or more targets in the biological sample;
performing image subtraction using the pre-PCR image and the post-PCR image.

32. The method of claim 31, wherein performing image subtraction using the pre-PCR image and the post-PCR image comprises one or more of:
mitigating or removing an artifact associated with image defects in both the pre-PCR image and the post-PCR image; and
mitigating or removing an artifact associated with contamination represented in both the pre-PCR image and the post-PCR image.

33. The method of any of claims 21-32, wherein quantifying the one or more target concentrations in the biological sample based on the observed locations of the partitions comprises:
providing images representing partitions associated with the observed locations of the partitions to a trained machine-learning model;
classifying, using the trained machine-learning model, the images as positive partition images or non-positive partition images; and
quantifying, based on a classification result of the classification of at least some of the partition images, the one or more target concentrations in the biological sample.

34. A non-transitory computer readable medium comprising a memory storing one or more instructions which, when executed by one or more processors of at least one computing device, quantify one or more target concentrations in a biological sample using an analyte detection apparatus configured to analyze an array of partitions of the biological sample by:
calculating expected locations of partitions in the array of partitions based on corner locations of the array of partitions;
analyzing images representing partitions associated with the expected locations of the partitions;
determining observed locations of the partitions based on an analysis result of the images; and
quantifying the one or more target concentrations in the biological sample based on the observed locations of the partitions.

35. A system for quantifying one or more target concentrations in a biological sample using an analyte detection apparatus configured to analyze an array of partitions of the biological sample, the system comprises:
one or more processors of at least one computing device; and
a memory storing one or more instructions, when executed by the one or more processors, cause the one or more processors to:
calculate expected locations of partitions in the array of partitions based on corner locations of the array of partitions;
analyze images representing partitions associated with the expected locations of the partitions;
determine observed locations of the partitions based on an analysis result of the images; and
quantify the one or more target concentrations in the biological sample based on the observed locations of the partitions.

36. A method of training a machine-learning model used for analyzing one or more biological samples by an analyte detection apparatus, the method being performed by one or more computing devices and comprising:
obtaining a first plurality of images identified as positive partition images;
obtaining a second plurality of images identified as non-positive partition images, the second plurality of images comprising one or more images modified from one or more other images identified as non-positive partition images;
generating one or more datasets using the first plurality of images and the second plurality of images; and
determining, by the one or more computing devices, a set of parameters of the machine-learning model by training the machine-learning model using at least one of the one or more datasets,
wherein a trained machine-learning model is configured based on the set of parameters to analyze one or more target concentrations in the one or more biological samples.

37. The method of claim 36, wherein the positive partition images are associated with positive partitions, wherein the positive partitions correspond to an existence of one or more target concentrations in the positive partitions.

38. The method of any of claims 36-37, wherein the non-positive partition images comprise at least one negative partition image associated with a negative partition, wherein the negative partition corresponds to a non-existence of a target concentration in the negative partition.

39. The method of any of claims 36-38, wherein the non-positive partition images comprise at least one image associated with a defective microchamber of a partition.

40. The method of any of claims 36-39, wherein the non-positive partition images comprise at least one image associated with a contaminated partition.

41. The method of any of claims 36-40, wherein the non-positive partition images comprise at least one image associated with a defective filling of a microchamber of a partition.

42. The method of any of claims 36-41, wherein the non-positive partition images comprise at least one defective image of a partition.

43. The method of claim any of claims 36-42, wherein obtaining the second plurality of images comprises:
obtaining a first subset of the second plurality of images identified as non-positive partition images;
modifying the first subset of the second plurality of images to obtain a second subset of the second plurality of images, the second subset being identified as non-positive partition images; and
including the second subset in the second plurality of images.

44. The method of claim 43, wherein modifying the first subset of the second plurality of images comprises one or more of rotating, editing, cropping, distorting, mirroring, brightening, darkening, changing a contrast of, changing a color of, and changing a pattern of, the first subset of the second plurality of images.

45. The method of claim any of claims 36-42, wherein obtaining the second plurality of images comprises:
obtaining a first subset of the first plurality of images identified as positive partition images;
modifying the first subset of the first plurality of images to obtain a third subset of the second plurality of images, the third subset being identified as non-positive partition images; and
including the third subset in the second plurality of images.

46. The method of claim 45, wherein modifying the first subset of the first plurality of images comprises one or more of rotating, editing, cropping, distorting, mirroring, brightening, darkening, changing a contrast of, changing a color of, and changing a pattern of, the first subset of the first plurality of images.

47. The method of claim any of claims 36-46, wherein the one or more datasets comprise a training dataset, a validation dataset, and a testing dataset.

48. The method of any of claims 36-47, wherein determining the set of parameters of the machine-learning model comprises:
iteratively training the machine-learning model using the one or more datasets; and
determining the set of parameters of the machine-learning model based on a result of the iterative training.

49. The method of any of claims 36-48, wherein the machine-learning model comprises a convolutional neural network (CNN).

50. A method for quantifying one or more target concentrations in a biological sample using an analyte detection apparatus configured to analyze an array of partitions of the biological sample, the method comprising:
providing a plurality of partition images to a trained machine-learning model, the plurality of partition images representing a corresponding plurality of partitions that are at least a subset of the array of partitions;
classifying, by the trained machine-learning model, the plurality of partition images as positive partition images or non-positive partition images, wherein the trained machine-learning model is trained by using one or more images modified from one or more other images identified as non-positive partition images; and
quantifying the one or more target concentrations in the biological sample based on a classification result.

51. The method of claim 50, further comprising:
determining, based on an image representing the array of partitions of the biological sample, a plurality of partition locations of the plurality of partitions; and
obtaining the plurality of partition images based on the corresponding plurality of partition locations.

52. The method of claim 51, further comprising, prior to determining the plurality of partition locations of the plurality of partitions:
obtaining a pre-PCR image representing the plurality of partitions before amplification of one or more targets in the biological sample;
obtaining a post-PCR image representing the plurality of partitions after amplification of the one or more targets in the biological sample; and
performing image subtraction using the pre-PCR image and the post-PCR image to obtain the plurality of partition images provided to the trained machine-learning model.

53. The method of claim 52, wherein performing image subtraction using the pre-PCR image and the post-PCR image comprises one or more of:
mitigating or removing an artifact associated with image defects in both the pre-PCR image and the post-PCR image; and
mitigating or removing an artifact associated with contamination represented in both the pre-PCR image and the post-PCR image.

54. The method of claim 53, wherein classifying, by the trained machine-learning model, the plurality of partition images as positive partition images or non-positive partition images comprises, for each partition image of the plurality of partition images:
determining a probability that the partition image is a positive partition image; and
classifying, based on the probability, the partition image as a positive partition image or a non-positive partition image.

55. The method of claim any of claims 50-54, wherein quantifying the one or more target concentrations in the biological sample comprises:
processing the classification result based on a threshold of positive partition images; and
quantifying, based on the processed classification result, the one or more target concentrations in the biological sample.

56. A non-transitory computer readable medium comprising a memory storing one or more instructions which, when executed by one or more processors of at least one computing device, perform training of a machine-learning model used for analyzing one or more biological samples by an analyte detection apparatus by performing processing comprising:
obtaining a first plurality of images identified as positive partition images;
obtaining a second plurality of images identified as non-positive partition images, the second plurality of images comprising one or more images modified from one or more other images identified as non-positive partition images;
generating one or more datasets using the first plurality of images and the second plurality of images; and
determining, by the one or more computing devices, a set of parameters of the machine-learning model by training the machine-learning model using at least one of the one or more datasets,
wherein a trained machine-learning model is configured based on the set of parameters to analyze one or more target concentrations in the one or more biological samples.

57. A non-transitory computer readable medium comprising a memory storing one or more instructions which, when executed by one or more processors of at least one computing device, perform a quantification of one or more target concentrations in a biological sample using an analyte detection apparatus configured to analyze an array of partitions of the biological sample by:
providing a plurality of partition images to a trained machine-learning model, the plurality of partition images representing a corresponding plurality of partitions that are at least a subset of the array of partitions;
classifying, by the trained machine-learning model, the plurality of partition images as positive partition images or non-positive partition images, wherein the trained machine-learning model is trained by using one or more images modified from one or more other images identified as non-positive partition images; and
quantifying the one or more target concentrations in the biological sample based on a classification result.

58. A system for training a machine-learning model used for analyzing one or more biological samples by an analyte detection apparatus, the system comprising:
one or more processors of at least one computing device; and
a memory storing one or more instructions, which, when executed by the one or more processors, cause the one or more processors to perform processing comprising:
obtaining a first plurality of images identified as positive partition images;
obtaining a second plurality of images identified as non-positive partition images, the second plurality of images comprising one or more images modified from one or more other images identified as non-positive partition images;
generating one or more datasets using the first plurality of images and the second plurality of images; and
determining, by the one or more computing devices, a set of parameters of the machine-learning model by training the machine-learning model using at least one of the one or more datasets,
wherein a trained machine-learning model is configured based on the set of parameters to analyze one or more target concentrations in the one or more biological samples.

59. A system for quantifying one or more target concentrations in a biological sample using an analyte detection apparatus configured to analyze an array of partitions of the biological sample, the system comprising:
one or more processors of at least one computing device; and
a memory storing one or more instructions, which, when executed by the one or more processors, cause the one or more processors to perform processing comprising:
providing a plurality of partition images to a trained machine-learning model, the plurality of partition images representing a corresponding plurality of partitions that are at least a subset of the array of partitions;
classifying, by the trained machine-learning model, the plurality of partition images as positive partition images or non-positive partition images, wherein the trained machine-learning model is trained by using one or more images modified from one or more other images identified as non-positive partition images; and
quantifying the one or more target concentrations in the biological sample based on a classification result.
